SEQUENCE LISTING (35 sequences)

SEQ ID NO: 1 - protein, 39 aa, synthetic construct
  MOD_RES 39: note = c-terminal amidation (-NH2)
HGEGTFTSDLSKQMEEEAVRLFIEWLKNGGPSSGAPPPS

SEQ ID NO: 2 - protein, 40 aa, synthetic construct
  MOD_RES 40: note = c-terminal amidation (-NH2)
HGEGTFTSDLSKQMEEEAVRLFIEWLKNGGPSSGAPPPSC

SEQ ID NO: 3 - protein, 40 aa, synthetic construct
  MOD_RES 40: note = c-terminal amidation (-NH2)
HGEGTFTSDLSKQMEEEAVRLFIEWLKNGGPSSGAPPPSK

SEQ ID NO: 4 - protein, 42 aa, synthetic construct
  MOD_RES 42: note = c-terminal amidation (-NH2)
HGEGTFTSDLSKQMEEEAVRLFIEWLKNGGPSSGAPPPSKKK

SEQ ID NO: 5 - protein, 40 aa, synthetic construct
  MOD_RES 40: note = c-terminal amidation (-NH2)
  SITE 2: note = X is Aib
HXEGTFTSDLSKQMEEEAVRLFIEWLKNGGPSSGAPPPSK

SEQ ID NO: 6 - protein, 42 aa, synthetic construct
  MOD_RES 42: note = c-terminal amidation (-NH2)
  SITE 2: note = X is Aib
HXEGTFTSDLSKQMEEEAVRLFIEWLKNGGPSSGAPPPSKKK

SEQ ID NO: 7 - protein, 40 aa, synthetic construct
  MOD_RES 40: note = c-terminal amidation (-NH2)
  SITE 2: note = X is Aib
HXEGTFTSDLSKQMEEEAVRLFIEWLKNGGPSSGAPPPSC

SEQ ID NO: 8 - protein, 40 aa, synthetic construct
  MOD_RES 40: note = c-terminal amidation (-NH2)
  SITE 1: note = X is des-amino-Histidine
  SITE 2: note = X is Aib
XXEGTFTSDLSKQMEEEAVRLFIEWLKNGGPSSGAPPPSC

SEQ ID NO: 9 - protein, 40 aa, synthetic construct; identical to SEQ ID NO: 2
  MOD_RES 40: note = N-Biotinoyl-Nprime-(6-maleimidohexanoyl)hydrazide
  MOD_RES 40: note = c-terminal amidation (-NH2)

SEQ ID NO: 10 - protein, 40 aa, synthetic construct; identical to SEQ ID NO: 2
  MOD_RES 40: note = 3-Maleimidopropionate-Lys(Biotin)-Lys(Biotin)-CONH2
  MOD_RES 40: note = c-terminal amidation (-NH2)

SEQ ID NO: 11 - protein, 40 aa, synthetic construct; identical to SEQ ID NO: 2
  MOD_RES 40: note = 3-Maleimidopropionate-Lys(Biotin)-Lys(Biotin)-Lys(B iotin)-CONH2
  MOD_RES 40: note = c-terminal amidation (-NH2)

SEQ ID NO: 12 - protein, 39 aa, synthetic construct; identical to SEQ ID NO: 1
  MOD_RES 27: note = propionate-N-hydroxysuccinimide ester- PEG-Lys(Biotin)-Lys(Biotin)-Lys(Biotin)-CONH2
  MOD_RES 39: note = c-terminal amidation (-NH2)

SEQ ID NO: 13 - protein, 40 aa, synthetic construct; identical to SEQ ID NO: 2
  MOD_RES 40: note = 3-Maleimidopropionate-PEG-Lys(Biotin)-Lys(Biotin)-L ys(Biotin)-CONH2
  MOD_RES 40: note = c-terminal amidation (-NH2)

SEQ ID NO: 14 - protein, 40 aa, synthetic construct; identical to SEQ ID NO: 2
  MOD_RES 40: note = 3-Maleimidopropionate-Lys(Biotin)-Lys(Biotin)-Lys(B iotin)-CONH2
  MOD_RES 27: note = C16-NHS
  MOD_RES 40: note = c-terminal amidation (-NH2)

SEQ ID NO: 15 - protein, 40 aa, synthetic construct; identical to SEQ ID NO: 2
  MOD_RES 12: note = N-hydroxysuccinimide-lysine
  MOD_RES 27: note = N-hydroxysuccinimide-lysine
  MOD_RES 40: note = C16-MAL
  MOD_RES 40: note = c-terminal amidation (-NH2)

SEQ ID NO: 16 - protein, 40 aa, synthetic construct; identical to SEQ ID NO: 2
  MOD_RES 40: note = 3-Maleimidopropionate-Lys(Biotin)-Lys(Biotin)-Lys(B iotin)-CONH2
  MOD_RES 27: note = C18-gamma Glu-2OEG-NHS
  MOD_RES 40: note = c-terminal amidation (-NH2)

SEQ ID NO: 17 - protein, 40 aa, synthetic construct; identical to SEQ ID NO: 2
  MOD_RES 12: note = (N-hydroxysuccinimide)-lysine
  MOD_RES 27: note = (N-hydroxysuccinimide)-lysine
  MOD_RES 40: note = C18-gamma Glu-2OEG-MAL
  MOD_RES 40: note = c-terminal amidation (-NH2)

SEQ ID NO: 18 - protein, 40 aa, synthetic construct; identical to SEQ ID NO: 2
  MOD_RES 40: note = Maleimide-(C18-gamma Glu-2OEG)-lysine
  MOD_RES 40: note = c-terminal amidation (-NH2)

SEQ ID NO: 19 - protein, 40 aa, synthetic construct; identical to SEQ ID NO: 2
  MOD_RES 40: note = Maleimide-(C18-gammaGlu-2OEG)-lysine
  MOD_RES 40: note = c-terminal amidation (-NH2)

SEQ ID NO: 20 - protein, 40 aa, synthetic construct; identical to SEQ ID NO: 3
  MOD_RES 40: note = Amine-(EG2-NH-(CH2)5-COOH)-lysine
  MOD_RES 40: note = c-terminal amidation (-NH2)

SEQ ID NO: 21 - protein, 42 aa, synthetic construct; identical to SEQ ID NO: 4
  MOD_RES 39: note = Biotin moiety (Chemical Formula A-1 in application)
  MOD_RES 40: note = Biotin moiety (Chemical Formula A-1 in application)
  MOD_RES 41: note = Biotin moiety (Chemical Formula A-1 in application)
  MOD_RES 42: note = c-terminal amidation (-NH2)

SEQ ID NO: 22 - protein, 42 aa, synthetic construct; identical to SEQ ID NO: 4
  MOD_RES 39: note = Biotin moiety (Chemical Formula A-1 in application)
  MOD_RES 40: note = Biotin moiety (Chemical Formula A-1 in application)
  MOD_RES 41: note = Biotin moiety (Chemical Formula A-1 in application)
  MOD_RES 27: note = C18-gammaGlu-2OEG-NHS
  MOD_RES 42: note = c-terminal amidation (-NH2)

SEQ ID NO: 23 - protein, 42 aa, synthetic construct; identical to SEQ ID NO: 4
  MOD_RES 39: note = Biotin moiety (Chemical Formula A-1 in application)
  MOD_RES 40: note = Biotin moiety (Chemical Formula A-1 in application)
  MOD_RES 41: note = Biotin moiety (Chemical Formula A-1 in application)
  MOD_RES 12: note = C18-gammaGlu-2OEG-NHS
  MOD_RES 42: note = c-terminal amidation (-NH2)

SEQ ID NO: 24 - protein, 40 aa, synthetic construct; identical to SEQ ID NO: 5
  MOD_RES 40: note = Amine-(EG2-NH-(CH2)5-COOH)-Lysine
  MOD_RES 40: note = c-terminal amidation (-NH2)
  SITE 2: note = X is Aib

SEQ ID NO: 25 - protein, 42 aa, synthetic construct; identical to SEQ ID NO: 6
  MOD_RES 39: note = Biotin moiety (Chemical Formula A-1 in application)
  MOD_RES 40: note = Biotin moiety (Chemical Formula A-1 in application)
  MOD_RES 41: note = Biotin moiety (Chemical Formula A-1 in application)
  MOD_RES 42: note = c-terminal amidation (-NH2)
  SITE 2: note = X is Aib

SEQ ID NO: 26 - protein, 42 aa, synthetic construct; identical to SEQ ID NO: 6
  MOD_RES 39: note = Biotin moiety (Chemical Formula A-1 in application)
  MOD_RES 40: note = Biotin moiety (Chemical Formula A-1 in application)
  MOD_RES 41: note = Biotin moiety (Chemical Formula A-1 in application)
  MOD_RES 27: note = C18-gammaGlu-2OEG-NHS
  MOD_RES 42: note = c-terminal amidation (-NH2)
  SITE 2: note = X is Aib

SEQ ID NO: 27 - protein, 42 aa, synthetic construct; identical to SEQ ID NO: 6
  MOD_RES 39: note = Biotin moiety (Chemical Formula A-1 in application)
  MOD_RES 40: note = Biotin moiety (Chemical Formula A-1 in application)
  MOD_RES 41: note = Biotin moiety (Chemical Formula A-1 in application)
  MOD_RES 12: note = C18-gammaGlu-2OEG-NHS
  MOD_RES 42: note = c-terminal amidation (-NH2)
  SITE 2: note = X is Aib

SEQ ID NO: 28 - protein, 40 aa, synthetic construct; identical to SEQ ID NO: 5
  MOD_RES 40: note = Amine-(EG2-NH-(CH2)5-COOH)-Lysine
  MOD_RES 27: note = C18-gammaGlu-2OEG-MAL
  MOD_RES 40: note = c-terminal amidation (-NH2)
  SITE 2: note = X is Aib

SEQ ID NO: 29 - protein, 40 aa, synthetic construct; identical to SEQ ID NO: 5
  MOD_RES 12: note = N-hydroxysuccinimide-lysine
  MOD_RES 27: note = N-hydroxysuccinimide-Lysine
  MOD_RES 40: note = C18-gammaGlu-2OEG-NHS
  MOD_RES 40: note = c-terminal amidation (-NH2)
  SITE 2: note = X is Aib

SEQ ID NO: 30 - protein, 40 aa, synthetic construct; identical to SEQ ID NO: 7
  MOD_RES 40: note = C18-gammaGlu-2OEG-MAL
  MOD_RES 12: note = N-hydroxysuccinimide-Lysine
  MOD_RES 27: note = N-hydroxysuccinimide-Lysine
  MOD_RES 40: note = c-terminal amidation (-NH2)
  SITE 2: note = X is Aib

SEQ ID NO: 31 - protein, 40 aa, synthetic construct; identical to SEQ ID NO: 8
  MOD_RES 12: note = N-hydroxysuccinimide-Lysine
  MOD_RES 27: note = N-hydroxysuccinimide-Lysine
  MOD_RES 40: note = C18-gammaGlu-2OEG-MAL
  MOD_RES 40: note = c-terminal amidation (-NH2)
  SITE 2: note = X is Aib
  SITE 1: note = X is des-amino-Histidine

SEQ ID NO: 32 - protein, 39 aa, synthetic construct; identical to SEQ ID NO: 1
  MOD_RES 12: note = N-hydroxysuccinimide-Lysine
  MOD_RES 27: note = N-hydroxysuccinimide-Lysine
  MOD_RES 39: note = c-terminal amidation (-NH2)

SEQ ID NO: 33 - protein, 40 aa, synthetic construct; identical to SEQ ID NO: 2
  MOD_RES 40: note = C16-NHS
  MOD_RES 40: note = c-terminal amidation (-NH2)

SEQ ID NO: 34 - protein, 40 aa, synthetic construct; identical to SEQ ID NO: 2
  MOD_RES 40: note = C18-gammaGlu-2OEG-MAL
  MOD_RES 40: note = c-terminal amidation (-NH2)

SEQ ID NO: 35 - protein, 40 aa, synthetic construct; identical to SEQ ID NO: 2
  MOD_RES 12: note = N-hydroxysuccinimide-Lysine
  MOD_RES 27: note = N-hydroxysuccinimide-Lysine
  MOD_RES 40: note = C18-gammaGlu-2OEG-MAL
  MOD_RES 40: note = c-terminal amidation (-NH2)

(19) United States
(12) Patent Application Publication
(10) Pub. No.: US 2026/0124278 A1
(43) Pub. Date: May 7, 2026

(54) COMPOSITIONS AND METHODS FOR TREATMENT OF NEUROLOGICAL DISORDERS

(71) Applicant: D&D Pharmatech Inc., Gyeonggi-do (KR)

(72) Inventors: Eun Ji PARK, Seoul (KR); Jong-Sung PARK, Gyeonggi-do (KR)

(21) Appl. No.: 18/869,210

(22) PCT Filed: May 26, 2023
(86) PCT No.: PCT/IB2023/055432

§ 371 Date: Nov. 25, 2024

Related U.S. Application Data
(60) Provisional application No. 63/346,502, filed on May 27, 2022.

(51) Int. Cl. A61K 38/26; A61K 47/54; A61K 47/55; A61P 25/16; A61P 25/28

(52) U.S. Cl. CPC A61K 38/26; A61K 47/542; A61K 47/551; A61P 25/16; A61P 25/28

(57) ABSTRACT

Long-acting glucagon like peptide 1 receptor agonists (GLP-1r agonists) reduce and inhibit pathological processes that give rise to long-term neurological impairment. A biotinylated and/or lipidated GLP-1r agonist analogs with enhanced enzymatic stability needed for gastrointestinal absorption, improved bioavailability and pharmacokinetics are described. In preferred embodiments, the GLP-1r agonist analogs have the amino acid sequence of any one of SEQ ID NOs: 9-35. The compositions are typically administered via oral or parenteral routes. The compositions are particularly suited for treating, alleviating, and/or preventing one or more neurological diseases or disorders such as Alzheimer's disease (AD) and Parkinson's disease (PD). Methods of treating a human subject having AD or PD or at risk of AD or PD are provided.

### CROSS-REFERENCE TO RELATED APPLICATION

This application is a National Phase application under 35 U.S.C. 371 of PCT/IB2023/055432, filed May 26, 2023, which claims the benefit of and priority to U.S. Ser. No. 63/346,502 filed May 27, 2022, and which are incorporated by reference herein in their entirety.

### REFERENCE TO SEQUENCE LISTING

The Sequence Listing submitted as an XML file named “DDP_106_PCT_ST26.xml” created on Nov. 4, 2025, and having a size of 74,340 bytes is hereby incorporated by reference pursuant to 37 C.F.R. § 1.834 (c) (1).

### FIELD OF THE INVENTION

The invention is generally in the field of treatment for neurological disorders, and in particular, methods and compositions for treating Parkinson's disease and Alzheimer's disease.

### BACKGROUND OF THE INVENTION

Neurodegenerative disease encompasses a range of conditions induced by the progressive loss of structure or function of neurons, including death of neurons. Diverse neurodegenerative diseases including Parkinson's disease (PD), Alzheimer's disease (AD), amyotrophic lateral sclerosis (ALS) and Huntington's disease (HD) occur because of neurodegenerative processes.

Parkinson's disease (PD) is a late onset, progressive neurodegenerative disorder that affects about one million Americans and 7 to 10 million people worldwide. Although dopamine replacement alleviates the symptomatic motor dysfunction, its effectiveness is reduced as the disease progresses, leading to unacceptable side effects such as severe motor fluctuations and dyskinesias. Moreover, this palliative therapeutic approach does not address the underlying mechanisms of the disease.

Alzheimer's disease (AD) is one of the most common neurodegenerative disease and accounts for more than 80% of dementia cases worldwide. It leads to the progressive loss of mental, behavioral, functional decline and ability to learn. In 2020, an estimated 5.8 million Americans aged 65 years or older had Alzheimer's disease. This number is projected to nearly triple to 14 million people by 2060 (Matthews, K. A. et al., Alzheimer's & Dementia (2018)). Currently approved treatments, e.g., acetylcholinesterase inhibitors, only provide symptomatic improvement but do not modify the disease process. The number of new strategies including the amyloid and tau-based therapeutics are in clinical development, however, no drugs have proven to show clear efficacy in humans.

Thus, despite significant efforts, no effective therapeutic agents or treatment methods have been approved to repair, or counteract, the neuronal damage of these neurodegenerative diseases, or the associated cognitive decline or impairment. New disease modifying treatments are sorely needed.

Therefore, it is an object of the invention to provide compositions and methods to reduce or prevent the pathological processes associated with the development and progression of neurological diseases such as Parkinson's disease and Alzheimer's disease, and methods of making and using thereof.

It is also an object of the invention to provide compositions and methods for the treatment or prevention of neuronal damage associated with Parkinson's disease and Alzheimer's disease and the associated motor and cognitive decline or impairment.

It is yet a further object of the invention to provide compositions and methods for blocking or reducing microglial activation and/or reactive astrocytes in neurodegenerative diseases with minimal off-target toxicity.

### SUMMARY OF THE INVENTION

It has been established that long-acting glucagon like peptide 1 receptor agonists (long-acting GLP-1r agonists) reduce and/or inhibit pathological processes such as microglial activation caused by abnormally aggregated proteins such as α-synuclein, β-amyloid or tau. While GLP-1r agonists are effective in neurodegenerative disease through inhibition of microglial activation, currently available GLP-1r agonists delivery are suboptimal; subcutaneous injection lowers patient compliance and oral administration has lower bioavailability due to enzymatic instability and insufficient gastrointestinal absorption because of peptide's inherent properties. Modified GLP-1r agonists show improved enzymatic stability, higher oral bioavailability and increased blood brain barrier (BBB) permeation.

Modified GLP-1r agonists with improved pharmacokinetic properties share the same target receptor and should have the same effects on microglia, though their clinical efficacy may not be the same and may differ depending on tissue distribution, blood brain barrier access, receptor kinetics, and exposure at well-tolerated doses.

Compositions and methods for treating or preventing a neurodegenerative disease or disorder in a subject suffering from or at risk of developing a neurodegenerative disease or disorder are provided. A composition including a long-acting GLP-1r agonist is administered in an amount effective to alleviate or treat one or more symptoms of the neurodegenerative disease or disorder. In some embodiments, the long-acting GLP-1r agonist is a polypeptide having the amino acid sequence of any one of SEQ ID NOs: 1-8 having conjugated thereto one or more biotin moieties and/or one or more fatty acids, or derivatives thereof, preferably via one or more cysteine and lysine residues. In preferred embodiments, one or more biotin moieties and/or one or more fatty acids, or derivative thereof, are conjugated to the polypeptide having the amino acid sequence of any one of SEQ ID NOs: 1-8 via amino acid residues lysine at position 12, lysine at position 27, and/or one or more C-terminal cysteine or lysine residue(s). In other embodiments, one or more cysteine and lysine residues are introduced via substitution or insertion into the amino acid sequence of any one of SEQ ID NOs: 1-8 to facilitate conjugation to one or more biotin moieties and/or one or more fatty acids, or derivatives thereof. In particular embodiments, the long-acting GLP-1r agonist has the amino acid sequence of any one of SEQ ID NOs: 9-35.

Typically, the methods administer an effective amount of the long-acting GLP-1r agonists to inhibit the secretion of inflammatory and/or neurotoxic mediators secreted from activated microglial cells and/or astrocytes. In some embodiments, the methods administer an effective amount of the long-acting GLP-1r agonists to reduce inflammatory or neurotoxic mediators selected from TNF-α, IL-1α, IL-1β, IFN-γ, IL-6, and C1q, as compared to an appropriate control. Preferably, the methods effectively reduce the number of activated microglial cells and/or reactive astrocytes in the brain of the subject, and/or reduce or inhibit the formation of abnormally aggregated proteins, such a α-synuclein, β-amyloid or tau through upregulation of GLP-1r. Generally, the methods are suitable for treating or preventing one or more symptoms of Parkinson's disease or Alzheimer's disease or other neurodegenerative disease in a subject in need thereof.

In some embodiments, the composition is administered via oral administration or parenteral administration, preferentially as subcutaneous administration. In preferred embodiments, the composition is orally administered in a form of pills, capsules, tablets, liquids, or suspensions.

Typically, the composition is administered at an interval of once a month, once every two weeks, once a week, once every three days, once every two days, once daily, or twice daily, for a duration of between one and 10 days, weeks, months, or years, inclusive. In some embodiments, the composition is administered to a human subject at a dose of between 0.001 mg/kg body weight of the subject and 100 mg/kg body weight of the subject, inclusive, or at a dose of between 1.0 mg and 100 mg, inclusive.

### BRIEF DESCRIPTION OF THE DRAWINGS

FIGS. 1A-1D are schematic illustrations of GLP-1r agonists with exemplary modifications including amino acid substitution, addition of biotin moieties, and addition of lipid molecules, for improvement in one or more of the biochemical properties of the peptide. FIG. 1A is a schematic showing exemplary 2-aminoisobutyric acid (Aib) substitution at amino acid position 2, exemplary addition of biotin moieties to Lysine residues at positions 12 and 27, and exemplary addition of a lipid moiety to C-terminal amino acid Lysine at position 40. FIG. 1B is a schematic showing a GLP-1r agonist having SEQ ID NO:29. FIG. 1C is a schematic showing a GLP-1r agonist having SEQ ID NO: 17. FIG. 1D is a schematic showing a GLP-1r agonist having SEQ ID NO:11.

FIG. 2 is a schematic diagram showing treatment of microglia with α-syn PFF in the presence of a GLP-1r agonist (e.g., DD02S), subsequent treatment of astrocytes with α-syn PFF microglial-conditioned medium (MCM); and subsequent treatment of neurons using the astrocyte-conditioned medium (ACM) to probe the mechanism of action of the GLP-1r agonist.

FIGS. 3A-3E are bar graphs showing the relative levels of mRNA expression of TNFα (FIG. 3A), IL-1α (FIG. 3B), IL-1β (FIG. 3C), IL-6 (FIG. 3D), and C1q (FIG. 3E) in response to α-syn PFF in mouse microglia including a control group with no α-syn PFF (Ctrl), in an experimental group with α-syn PFF only (PFF), in experimental groups with α-syn PFF in the presence of a GLP-1r agonist, NLY01, Semaglutide, or DD02S. Data are mean±s.e.m.; n=3-6 biologically independent experiment; p values were determined by one-way ANOVA. **P<0.01, ****P<0.0001 versus vehicle control; #P<0.05, ##P<0.01, ###P<0.001, ####P<0.0001 versus α-syn PFF.

FIG. 4 is a bar graph showing the relative levels of mRNA expression of C3 in mouse astrocytes after culturing in α-syn PFF microglial-conditioned medium (MCM) treated with NLY01, Semaglutide, or DD02S, and followed by PFF 150 nM for 24 h. A control groups with no α-syn PFF (Ctrl) and an experimental group with α-syn PFF only (PFF) are also included. Data are mean±s.e.m.; n=3-6 biologically independent experiment; p values were determined by one-way ANOVA. **P<0.001 versus vehicle control; ##P<0.01 versus α-syn PFF.

FIGS. 5A-5D are bar graphs showing the relative levels of mRNA expression of TNFα (FIG. 5A), IL-1α (FIG. 5B), IL-1β (FIG. 5C), and IL-6 (FIG. 5D), in response to β-amyloid oligomer (ABO) in mouse microglia including a control group with no AB (Ctrl), in an experimental group with ABO only (A-beta), in experimental groups with ABO in the presence of a GLP-1r agonist, NLY01, Semaglutide, or DD02S. Data are mean±s.e.m.; n=3-6 biologically independent experiment; p values were determined by one-way ANOVA. ***P<0.001, ****P<0.0001 versus vehicle control; #P<0.05, ##P<0.01, ###P<0.001, ####P<0.0001 versus α-syn PFF.

FIGS. 6A-6E are bar graphs showing the relative levels of mRNA expression of C3 (FIG. 6A), LCN2 (FIG. 6B), GBP2 (FIG. 6C), Cxc110 (FIG. 6D), and Steap4 (FIG. 6E) in mouse astrocytes after culturing in ABO microglial-conditioned medium (MCM) treated with NLY01, Semaglutide, or DD02S, and followed by ABO 150 nM for 24 h. A control groups with no ABO (Ctrl) and an experimental group with ABO only (A-beta) are also included. Data are mean±s.e.m.; n=3-6 biologically independent experiment; p values were determined by one-way ANOVA. **P<0.01, ****P<0.0001 versus vehicle control; #P<0.05, ##P<0.01 versus α-syn PFF.

FIGS. 7A-7E are bar graphs showing the relative levels of mRNA expression of TNFα (FIG. 7A), IL-1α (FIG. 7B), IL-1β (FIG. 7C), C1q (FIG. 7D), and IL-6 (FIG. 7E) in response to β-amyloid oligomer (ABO) in mouse microglia including a control group with no ABO (Ctrl), in an experimental group with ABO only (A-beta), in experimental groups with ABO in the presence of a GLP-1r agonist, NLY01, Semaglutide, DD0205, DD0206, or DD0207; p values were determined by one-way ANOVA. *P<0.05, **P<0.001

FIGS. 8A and 8B are showing the intracellular uptake of biotinylated GLP-1r agonists, SEQ ID NO: 11 (FIG. 8A), SEQ ID NO:29, SEQ ID NO:35 and Exenatide (FIG. 8B) in Caco-2 cells.

FIGS. 9A and 9B are showing the quantification of immunoreactivity of pS129-α-syn in the STR (FIG. 9A) and SNpc (FIG. 9B) in α-syn PFF induced PD mice.

### DETAILED DESCRIPTION OF THE INVENTION

### I. Definitions

The term “activated microglial cells” refers to microglia, the resident immune cells of the CNS, which normally respond to neuronal damage and remove the damaged cells by phagocytosis. Under steady-state conditions, microglia are maintained in a “resting” state through interactions with cell surface and soluble factors from surrounding cells. In response to neurodegeneration and the accumulation of abnormally aggregated proteins, such as α-synuclein and β-amyloid, resting microglia become an activated state and release various cytokines and neurotoxic molecules, and activate astrocytes. Consequently, such inflammatory mediators released from activated microglia or reactive astrocytes, induced by activated microglia, causes neuronal damage and contribute to the progression of neurodegenerative diseases.

Neuroinflammation, defined as inflammation of nervous tissue, is initiated in response to a variety of endogenous and exogenous sources including invading pathogens, neuronal injury, and toxic compounds. It is characterized by glial cell activation, the release of inflammatory molecules, increased blood-brain barrier permeability, and recruitment of peripheral immune cells into the brain. The chronic activation of microglia may in turn cause neuronal damage through the release of potentially cytotoxic molecules such as proinflammatory cytokines, reactive oxygen intermediates, proteinases and complement proteins.

The term “therapeutic agent” refers to an agent that can be administered to treat one or more symptoms of a disease or disorder. The term “prophylactic agent” generally refers to an agent that can be administered to prevent disease or to prevent certain conditions.

The term “pharmaceutically acceptable salt”, as used herein, refers to derivatives of the compounds defined herein, wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. Pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “therapeutically effective amount” refers to an amount of the therapeutic agent that produces some desired effect at a reasonable benefit/risk ratio applicable to any medical treatment. The effective amount may vary depending on such factors as the disease or condition being treated, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish one or more symptoms of one or more diseases or disorders, such as reducing, preventing, or reversing the learning and/or memory deficits in an individual suffering from Alzheimer's disease etc. In one or more neurological or neurodegenerative diseases, an effective amount of the drug may have the effect of stimulation or induction of neural mitosis leading to the generation of new neurons, i.e., exhibiting a neurogenic effect; prevention or retardation of neural loss, including a decrease in the rate of neural loss, i.e., exhibiting a neuroprotective effect. An effective amount can be administered in one or more administrations.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, long-lasting GLP-1r agonists may inhibit or reduce the activity and/or quantity of activated microglia by about 10%, 20%, 30%, 40%, 50%, 75%, 85%, 90%, 95%, or 99% from the activity and/or quantity of the same cells in equivalent tissues of subjects that did not receive or were not treated with long-lasting GLP-1r agonists. In some embodiments, the inhibition and reduction are compared at mRNAs, proteins, cells, tissues, and organs levels. For example, an inhibition and reduction in the rate of neural loss, in the rate of decrease of brain weight, or in the rate of decrease of hippocampal volume, as compared to an untreated control subject.

As used herein, an “HAC6 inhibitor” is a compound which blocks HADC6 interaction with the leptin receptor and/or inhibits HDAC6 in the hypothalamus.

The term “treating” or “treatment” refers to amelioration, alleviation or reduction of one or more symptoms of a disease, disorder, or condition in an person who may be predisposed to the disease, disorder and/or condition but has not yet been diagnosed as having it; reducing disease symptoms, inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms of neurological impairment associated AD are mitigated or eliminated, including reducing the rate of neuronal loss, decreasing symptoms resulting from the disease, increasing the quality of life of those suffering from the disease, decreasing the dose of other medications required to treat the disease, delaying the progression of the disease, and/or prolonging survival of individuals. The term “ameliorate” refers to a decrease, suppression, attenuation, diminish, arrest, or stabilization of the development or progression of a disease.

The terms “prevent”, “prevention” or “preventing” mean to administer a composition or method to a subject or a system at risk for or having a predisposition for one or more symptom caused by a disease or disorder, to decrease the likelihood the subject will develop one or more symptoms of the disease or disorder, or to reduce the severity, duration, or time of onset of one or more symptoms of the disease or disorder.

The term “biodegradable” generally refers to a material that will degrade or erode under physiologic conditions to smaller units or chemical species that are capable of being metabolized, eliminated, or excreted by the subject. The degradation time is a function of composition and morphology.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide.

The term “long-acting GLP-1r agonist” refers to a glucagon like peptide 1 receptor agonists (GLP-1r agonist) which is effective as a GLP-1r agonist for at least one hour, at least six hours, at least twelve hours, at least one day, at least two days, at least one week, at least two weeks, at least one month, or at least two months.

The terms “PEGylation” and “PEGylated” refer to the process and product of both covalent and non-covalent attachment or amalgamation of polyoxyalkylene oxide polymers, preferably polyethylene glycol (PEG) chains, to molecules and macrostructures, such as a drug, therapeutic protein, or particle.

The terms “biotinylation” and “biotinylated” refer to the process and product of both covalent attachment of one or more biotin moieties or derivatives thereof to molecules and macrostructures, such as a therapeutic protein.

The terms “lipidation” and “lipidated” refer to the process and product of both covalent attachment of one or more fatty acid moieties or derivatives thereof to molecules and macrostructures, such as a therapeutic protein.

Use of the term “about” is intended to describe values either above or below the stated value in a range of approximate+/−10%; in other embodiments the values may range in value either above or below the stated value in a range of approximately +/−5%.

### II. Compositions

Microglia are engaged in signaling with neurons through cytokines and neurotransmitters as well as direct neurite contact. When activated, microglial communication may be impeded, leading directly to defects in memory function and neural editing. Microglia are key mediators of inflammation in the central nervous system and have been identified as targets in neurodegenerative diseases like Parkinson's and Alzheimer's disease. Protein aggregates that are indicative of these diseases were found to stimulate microglial activation, leading to a cascade of events including pro-inflammatory cytokine production, neurotoxic astrocyte formation, and ultimately lead to decline in cognitive processes such as memory or motor coordination, depending on the location of neurodegeneration.

Considering the roles of microglia and astrocytes in brain inflammatory responses, cognition, and neurodegeneration, they are key targets for therapeutic strategies against neurological and neurodegenerative diseases.

Compositions of long-lasting GLP-1r agonists, as well as analogs and derivatives thereof, and pharmaceutical formulations thereof are provided. The compositions activate GLP-1r to effectively treat and prevent one or more symptoms of neurological and neurodegenerative diseases. In preferred embodiments, the long-lasting GLP-1r agonists are lipidated and/or biotinylated GLP-1r agonists. The compositions and formulations of these long-lasting GLP-1r agonists or analogs thereof are effective to alleviate or prevent one or more symptoms of neurological and neurodegenerative diseases in subjects in need thereof.

### A. GLP-1R Agonists

Glucagon-like peptide-1 (GLP-1) agonists are also known as GLP-1 receptor agonists, incretin mimetics, or GLP-1 analogs. Long-acting GLP-1r agonists reduce and inhibit pathological processes such as microglial activation. Thus, GLP-1R agonists and derivative thereof targeting the same target receptor should have the same effects on microglia, though their clinical efficacy may not be the same and may differ depending on tissue distribution, blood brain barrier access, receptor kinetics, and exposure at well-tolerated doses.

Compositions and pharmaceutical formulations of long-acting GLP-1r agonists are provided.

An exemplary long-acting GLP-1r agonist is a GLP-1r agonist polypeptide (e.g., exenatide) that is modified to enhance plasma half-life, pharmacokinetics, and oral bioavailability. Exemplary GLP-1r agonists that can be modified include modified exenatide, dulaglutide and albiglutide. Exemplary modifications include substitution, addition, or deletion of one or more amino acid residues within the polypeptide, and/or addition of biotin moieties, and/or addition of one or more fatty acid chain(s) to the polypeptide. A preferred modified GLP-1r agonist is exenatide having an amino acid sequence of SEQ ID NO:1, conjugated to one or more biotin moieties and/or one or more fatty acids, optionally with one or more spacers, to achieve desired pharmacokinetics, stability, and bioavailability. A further preferred modified GLP-1r agonist is exenatide having an amino acid sequence of SEQ ID NO:1 conjugated to one or more fatty acids and/or one or more biotin moieties via an additional cysteine or lysine residue at the Carboxyl (C-)terminus, and to one or more internal lysine residue(s), preferably including 2-aminoisobutyric acid (Aib) at the Glycine (Gly; G) residue in the second position of the amino acid sequence of SEQ ID NO:1.

### 1. Exenatide

Exenatide (Exendin-4) is a peptide agonist of GLP-1R that facilitates insulin release in type two diabetes (T2D) and is marketed as BYETTA® for T2D (Meier, J J, Nat Rev Endocrinol, 2012. 8 (12): p. 728-42). Also known as “exendin-4” and marketed as “BYETTA®” and “BYDUREON®” exenatide is an engineered Glucagon-like peptide-1 receptor agonist peptide drug having CAS No. 141758-74-9. Exenatide is a 39-amino-acid peptide, an insulin secretagogue, with glucoregulatory effects. The peptide sequence of exenatide is: HGEGTFTSDLSKQMEEEAVRLFIEWLKNGGPSSGAPPPS (SEQ ID NO: 1).

This peptide drug manages insulin release in a glucose-dependent manner and is therefore safe for non-diabetic patients. Exenatide also reduces a range of neurodegenerative processes (Holscher, C., J Endocrinol, 2014. 221(1): p. T31-41). In preclinical models, exenatide crosses the blood brain barrier (BBB), protects memory formation in AD or motor activity in PD, protects synapses and synaptic functions, enhances neurogenesis, reduces apoptosis, protects neurons from oxidative stress, as well as reduces plaque formation and the chronic inflammation response in the brains of AD and PD mouse models. Exenatide, like other peptide drugs, is inherently short-lived and unstable in the blood stream and therefore requires frequent injections.

Exenatide belongs to the group of incretin mimetics, approved in April 2005 for the treatment of diabetes mellitus type 2. Exenatide in its BYETTA® form is administered as a subcutaneous injection (under the skin) of the abdomen, thigh, or arm, any time within the 60 minutes before the first and last meal of the day. Exenatide was approved by the FDA on Apr. 28, 2005, for patients whose diabetes was not well-controlled on other oral medication. The medication is injected subcutaneously twice per day using a filled pen-like device. A once-weekly injection has been approved as of Jan. 27, 2012, under the trademark BYDUREON®). It is manufactured by Amylin Pharmaceuticals and commercialized by Astrazeneca.

Exenatide is a synthetic version of Exendin-4, a hormone found in the saliva of the Gila monster. It displays biological properties similar to human glucagon-like peptide-1 (GLP-1), a regulator of glucose metabolism and insulin secretion. According to the package insert, exenatide enhances glucose-dependent insulin secretion by the pancreatic beta-cell, suppresses inappropriately elevated glucagon secretion, and slows gastric emptying, although the mechanism of action is still under study.

The incretin hormones GLP-1 and glucose-dependent insulinotropic peptide (GIP) are produced by the L and K endocrine cells of the intestine following ingestion of food. GLP-1 and GIP stimulate insulin secretion from the beta cells of the islets of Langerhans in the pancreas. Only GLP-1 causes insulin secretion in the diabetic state; however, GLP-1 itself is ineffective as a clinical treatment for diabetes as it has a very short half-life in vivo. Exenatide bears a 50% amino acid homology to GLP-1 and it has a longer half-life in vivo. Thus, it was tested for its ability to stimulate insulin secretion and lower blood glucose in mammals and was found to be effective in the diabetic state. In studies on rodents, it has also been shown to increase the number of beta cells in the pancreas.

Commercially, exenatide is produced by direct chemical synthesis. Historically, exenatide was discovered as Exendin-4, a protein naturally secreted in the saliva and concentrated in the tail of the Gila monster. Exendin-4 shares extensive homology and function with mammalian GLP-1 but has a therapeutic advantage in its resistance to degradation by DPP-IV (which breaks down GLP-1 in mammals) therefore allowing for a longer pharmacological half-life. The biochemical characteristics of Exendin-4 enabled consideration and development of exenatide as a diabetes mellitus treatment strategy. Subsequent clinical testing led to the discovery of the also desirable glucagon and appetite-suppressant effects.

In its twice daily BYETTA® form, exenatide raises insulin levels quickly (within about ten minutes of administration) with the insulin levels subsiding substantially over the next hour or two. A dose taken after meals has a much smaller effect on blood sugar than one taken beforehand. The effects on blood sugar diminish after six to eight hours. In its BYETTA® form, the medicine is available in two doses: 5 mcg. and 10 mcg. Treatment often begins with the 5 mcg. dosage, which is increased if adverse effects are not significant. Its once weekly BYDUREON® form is unaffected by the time between the injection and when meals are taken. BYDUREON® has the advantage of providing 24-hour coverage for blood sugar lowering, while BYETTA® has the advantage of providing better control of the blood sugar spike that occurs right after eating. Per the FDA label for BYDUREON®, BYDUREON® lowers HbA1c blood sugar by an average of 1.6%, while BYETTA® lowers it by an average of 0.9%. Both BYETTA® and BYDUREON® have similar weight loss benefits. Per the FDA approved BYDUREON® label, the levels of nausea are lower for BYDUREON®) patients than for BYETTA® patients.

NLY01, as a long-acting PEGylated form of exenatide, has an extended half-life of 12±4 days in humans and 88 hours in most primates, compared to BYETTA® exenatide (exendin-4 which has a 2-hour half-life) and liraglutide (which has a 13-hour half-life). NLY01 maintains its biological activity by a site specifically attached polyethylene glycol (PEG) molecule to exenatide (WO2013002580). Despite a large molecular weight poly(ethylene glycol) polymer (PEG, 50,000 Da) conjugated to the small exenatide peptide (˜4,000 Da), NLY01 shows similar pharmacological efficacy to exenatide in Parkinson's Disease (PD) and Alzheimer's Disease (AD). NLY01 traverses the blood-brain barrier (BBB) and modulates the activity of activated microglia. Due to its greater half-life and potency, this compound is suitable for a once-weekly, bi-monthly or once-monthly clinical dosing frequency. This dosing frequency is an improvement over the current twice daily treatment (exenatide, BYETTA®) or once-daily treatment (liraglutide, VICTOZA®).

2. GLP-1r Agonist Analogs and their Modifications

The direct use of native polypeptides as biopharmaceuticals is often limited by their very short systemic half-lives resulting from a rapid metabolism, enzymatic degradation, and, for smaller proteins and peptides, effective renal clearance. Modifications to exenatide such as semaglutide, liraglutide, and NLY01, have extended the half-life and pharmacokinetics of the active agent. Thus, further modifications are made to further improve the oral bioavailability, stability, and/or pharmacokinetics. In some embodiments, the GLP-1r agonist analogs have the amino acid sequence of any one of SEQ ID NOs: 1-8 as shown in Table 1. In preferred embodiments, the long-acting GLP-1r agonists are modified with one or more biotin moieties, one or more fatty acids, and/or one of more polyethylene glycols, optionally with one or more spacers, to achieve desired pharmacokinetics, stability, and bioavailability. In exemplary embodiments, the long-acting GLP-1r agonists disclosed herein are modified with C-terminal amidation. In particular embodiments, the GLP-1r agonist analogs having the amino acid sequence of any one of SEO ID NOS: 1-35, are amidated at the C terminus.

The choice of the suitable functional group for modifications is based on the type of available reactive group on the molecule that will be coupled to the biotin moieties and/or fatty acids. For proteins, typical reactive amino acids include lysine, cysteine, histidine, arginine, aspartic acid, glutamic acid, serine, threonine, tyrosine. The N-terminal amino group and the C-terminal carboxylic acid can also be used as a site-specific conjugation. In preferred embodiments, the reactive amino acids are lysine and cysteine.

In preferred embodiments, one or more biotin moieties and/or one or more fatty acids, or derivative thereof, are conjugated to the amino acid sequence of any one of SEQ ID NOs: 1-8 via amino acid residues lysine at position 12, lysine at position 27, and/or one or more C-terminal cysteine or lysine residues. In other embodiments, one or more cysteine and lysine residues are introduced via substitution or insertion into the amino acid sequence of any one of SEQ ID NOs: 1-8 to facilitate conjugation to biotin moieties and/or fatty acids, or derivatives thereof. In particular embodiments, the long-acting GLP-1r agonist has the amino acid sequence of any one of SEQ ID NOs: 9-35.

TABLE 1
Peptide sequences

| Peptide | | |
|---|---|---|
| No. | Name | Amino Acid Sequence |
| 5 | EX4 | HGEGTFTSDLSKQMEEEAVRLFIEWLKNGGPSSGAPPPS |
| | | (SEQ ID NO: 1) |
| 12 | EX4C | HGEGTFTSDLSKQMEEEAVRLFIEWLKNGGPSSGAPPPSC |
| | | (SEQ ID NO: 2) |
| 18 | EX4K | HGEGTFTSDLSKQMEEEAVRLFIEWLKNGGPSSGAPPPSK |
| | | (SEQ ID NO: 3) |
| 19 | EX4KKK | HGEGTFTSDLSKQMEEEAVRLFIEWLKNGGPSSGAPPPSKKK |
| | | (SEQ ID NO: 4) |
| 20 | EX4K(Aib) | H-Aib-EGTFTSDLSKQMEEEAVRLFIEWLKNGGPSSGAPPPSK |
| | | (SEQ ID NO: 5) |
| 21 | EX4KKK(Aib) | H-Aib-EGTFTSDLSKQMEEEAVRLFIEWLKNGGPSSGAPPPSKKK |
| | | (SEQ ID NO: 6) |
| 38 | EX01C | H-Aib-EGTFTSDLSKQMEEEAVRLFIEWLKNGGPSSGAPPPSC |
| | | (SEQ ID NO: 7) |
| 39 | EX02C | h^{#}-Aib-EGTFTSDLSKQMEEEAVRLFIEWLKNGGPSSGAPPPSC |
| | | (SEQ ID NO:8) |

*Aib is 2-aminoisobutyric acid (Aib)
^{#}h is des-amino-His

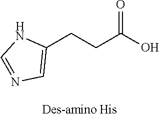

a. Biotinylation

Biotin modifications to exendin derivatives have been described, for example, in International Publication Nos. WO2009107900A1, WO2020242268A1, and WO2021107519A1.

Korean Patent Registration No. 10-0864584 describes an exendin-4 derivative in which biotin is modified in a lysine residue. This exendin-4 may be administered orally and the bioavailability in the intestine is improved. However, in this case, there is a problem in that biotin is conjugated to various lysine positions of exendin-4 to form various isomers, thereby lowering the reaction rate and yield, and biotin is conjugated to a lysine position of an N-terminal which is an active site of exendin-4 to inhibit the activity of exendin-4.

Accordingly, improved biotin modifications are needed for enhanced oral bioavailability without inhibiting the activity of exendin-4. Thus, in preferred embodiments, one or more biotin moieties are conjugated to amino acids (e.g., cysteine or lysine) at suitable positions to provide excellent oral bioavailability without inhibiting the activity of exendin-4.

In some embodiments, the biotin-conjugated GLP-1r agonists have an improved in vivo oral bioavailability compared to the same GLP-1r agonists without the one or more biotin moieties conjugated thereto. In preferred embodiments, the biotin-conjugated GLP-1r agonists retain most of the activity of the same GLP-1r agonists without the one or more biotin moieties conjugated thereto.

In some embodiments, the biotin moiety conjugated to one or more amino acid residues (e.g., cysteine or lysine) of a GLP-1r agonist is represented by the following General Formula A.

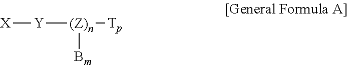

wherein,
X is a functional group capable of being conjugated to the polypeptide,
Y is a spacer,
Z is a binding unit,
B may be represented by the following Chemical Formula A-1,

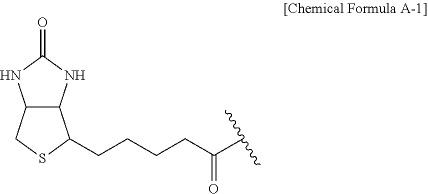 [SMILES: *C(=O)CCCCC1SCC2NC(=O)NC21]

T is a terminal group,
m is an integer of 1 to 10,
n is an integer of 1 to 10, and
p is an integer of 0 or 1.

In some embodiments, the biotin moiety-conjugated polypeptide is a peptide in which at least one of the amino acid residues at positions 9 to 39 of the amino acid sequence of SEQ ID NO:1 is substituted with cysteine or lysine. Here, the insertion means that cysteine or lysine is inserted before or after at least one of the amino acid residues at positions 9 to 39, inclusive. Alternatively, one or more cysteine or lysine residues are inserted internally at any position within the amino acid residues of SEQ ID NO: 1 to facilitate the conjugation to biotin.

In some embodiments, the biotin moiety is conjugated to the GLP-1r agonist polypeptide via one or more additional cysteine or lysine residues added to the C-terminus of the polypeptide. In one embodiment, the biotin moiety is conjugated to the GLP-1r agonist polypeptide via one additional cysteine residue added to the C-terminus of the amino acid sequence of SEQ ID NO: 1, and the resulting peptide has the amino acid sequence of SEQ ID NO: 2. In another embodiment, the biotin moiety is conjugated to the GLP-1r agonist polypeptide via one additional lysine residue added to the C-terminus of the amino acid sequence of SEQ ID NO: 1, and the resulting peptide has the amino acid sequence of SEQ ID NO:3. In yet another embodiment, one or more biotin moieties are conjugated to the GLP-1r agonist polypeptide via three additional lysine residues added to the C-terminus of the amino acid sequence of SEQ ID NO:1, and the resulting peptide has the amino acid sequence of SEQ ID NO:4. In some embodiments, the amino acid of the second position of SEQ ID NOs: 1-4 is substituted with 2-aminoisobutyric acid (Aib). In some embodiments, the biotin moiety is conjugated to the GLP-1r agonist polypeptide via one or more internal lysine residues, for example lysine at positions 12 and 27 of any of SEQ ID NOs: 1-8.

In General Formula A representing the biotin moiety, X is a functional group capable of being conjugated with cysteine of the polypeptide. Although not limited thereto, for example, the functional group may be maleimide, amine, succinimide, N-hydroxysuccinimide, aldehyde or carboxyl group, and more specifically maleimide.

In one embodiment, when the functional group X in General Formula A is conjugated with cysteine or lysine of the polypeptide, the structure may be maintained, or removed or modified.

In General Formula A, the Y may be a spacer and may have a structure having cleavability in the body. Although not limited thereto, for example, the Y is a direct-bonded, or substituted or unsubstituted alkylene, wherein the alkylene may include at least one of —O—, —C(═O) NR—, —C(═O)O— or —C(═O)—, —NR—, and —NOR—, and the R may be hydrogen, and substituted or unsubstituted alkyl or aryl.

In one embodiment, the spacer may include a structure represented by the following Formula.

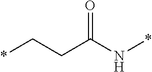 [SMILES: *CCC(=O)N*]

In some embodiments, in General Formula A, the Z is a binding unit capable of binding to B, and may include, for example, an amino acid, a polypeptide, an alkylene amine, or a polyamidoamine structure, but not limited thereto.

Although not limited thereto, for example, the amino acid may be lysine, 5-hydroxylysine, 4-oxallysine, 4-thialysine, 4-selenalysine, 4-thiahomolysine, 5,5-dimethyllysine, 5,5-difluorolysine, trans-4-dehydrolysine, 2,6-diamino-4-hexynoic acid, cis-4-dehydrolysine, 6-N-methyllysine, diminopimelic acid, ornithine, 3-methylornithine, α-methylornithine, citrulline or homocitrulline, arginine, aspartate, asparagine, glutamate, glutamine, histidine, ornithine, proline, serine, or threonine.

When the n is 0, B may directly bind to Y (spacer).

In some embodiments, in General Formula A, the T is a terminal group, and although not limited thereto, may be, for example, hydrogen or NH₂.

When the p is 0, the B may be a terminal.

In one embodiment, in General Formula A above, “m” may be an integer of 1 to 10, and specifically, may be an integer of 1 to 8, 1 to 5, and 1 to 4.

In one embodiment, the biotin moiety may be represented by the following General Formula 1A:

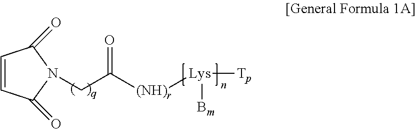

wherein,
Lys is lysine,
T is hydrogen or NH₂,
q is an integer of 1 to 5,
r is an integer of 0, 1 to 3, and
B, n, m, and p are as defined in General Formula A above.

In one embodiment, the biotin moiety may be represented by the following General Formula 2A or 3A:

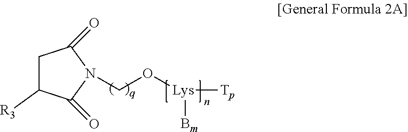

wherein,
Lys is lysine,
T is hydrogen or NH₂,
R₃ is hydrogen or —SO₃—,
q is an integer of 0, or 1 to 4, and
B, n, m, and p are as defined in General Formula A above.

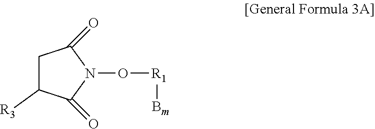

wherein,
R₁ is a direct bond or NH,
R₃ is hydrogen or —SO₃—, and
B and m are as defined in General Formula A above.

In one embodiment, the biotin moiety may be represented by the following structures I-III.

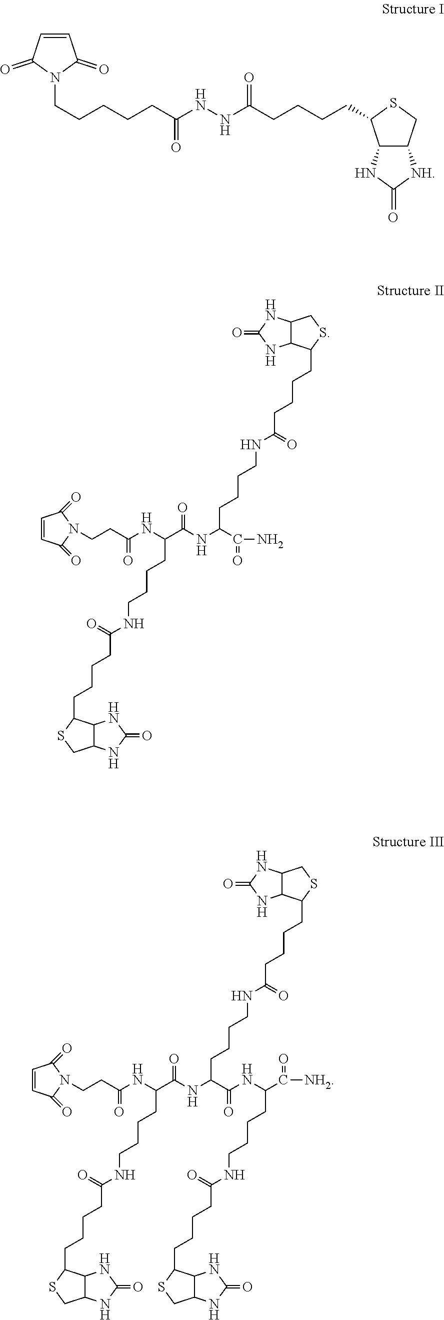 [SMILES: O=C(CCCCCN1C(=O)C=CC1=O)NNC(=O)CCCC[C@@H]1SC[C@@H]2NC(=O)N[C@@H]21 ; NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O ; NC(=O)C(CCCCNC(=O)CCCCC1SCC2NC(=O)NC21)NC(=O)C(CCCCNC(=O)CCCCC1SCC2NC(=O)NC21)NC(=O)C(CCCCNC(=O)CCCCC1SCC2NC(=O)NC21)NC(=O)CCN1C(=O)C=CC1=O]

Exemplary biotin derivatives are shown in Tables 2 and 3, below.

TABLE 2
Examples of biotin derivatives.

| Biotin | | |
|---|---|---|
| No. | Structure | Abbreviation |
| B1 | N-Biotinoyl-N′-(6-maleimidohexanoyl)hydrazide | B1-MAL |
| B2 | 3-Maleimidopropionate-Lys(Biotin)-Lys(Biotin)- | B2-MAL |
| | CONH₂ | |
| B3 | 3-Maleimidopropionate-Lys(Biotin)-Lys(Biotin)- | B3-MAL |
| | Lys(Biotin)-CONH₂ | |
| B4 | propionate-N-hydroxysuccinimide ester- PEG- | NHS- |
| | Lys(Biotin)-Lys(Biotin)-Lys(Biotin)-CONH₂ | PEG-B3 |
| B5 | 3-Maleimidopropionate-PEG-Lys(Biotin)- | MAL- |
| | Lys(Biotin)-Lys(Biotin)-CONH₂ | PEG-B3 |

TABLE 3
Examples of biotin derivatives.

| Biotin | | | | Number |
|---|---|---|---|---|
| No. | X | Y | Z | of Biotin |
| B6 | Aldehyde | propane | Lysine | 2 |
| B7 | Maleimide | butyrate | Glycerol and PEG | 2 |
| B8 | Maleimide | butyrate | Glycerol and PEG | 2 |
| B9 | N-hydroxysuccinimide | butyrate | Lysine | 2 |
| B10 | N-hydroxysuccinimide | glutarate | Glycerol and PEG | 2 |
| B11 | Maleimide | PEG12 | Lysine | 3 |
| B12 | N-hydroxysuccinimide | PEG12 | Lysine | 3 |
| B13 | amine | — | Lysine | 3 |
| B14 | Aldehyde | pentane | Lysine | 2 |
| B15 | Maleimide | adipate | Glycerol and PEG | 2 |
| B16 | Maleimide | suberate | Glycerol and PEG | 2 |
| B17 | Maleimide | sebacate | Glycerol and PEG | 2 |
| B18 | N-hydroxysuccinimide | adipate | Glycerol and PEG | 2 |
| B19 | N-hydroxysuccinimide | suberate | Lysine | 4 |
| B20 | N-hydroxysuccinimide | sebacate | Lysine | 4 |
| B21 | N-hydroxysuccinimide | PEG6 | Glycerol and PEG | 2 |
| B22 | Succinimidyl carbonate | PEG6 | Lysine | 2 |
| B23 | Succinimidyl carbonate | PEG12 | Lysine | 3 |
| B24 | Succinimidyl carbonate | pentane | Lysine | 3 |
| B25 | Succinimidyl carbonate | hexane | Lysine | 3 |
| B26 | p-nitrophenyl carbonate | PEG6 | Lysine | 3 |
| B27 | p-nitrophenyl carbonate | PEG12 | Lysine | 4 |
| B28 | p-nitrophenyl carbonate | propane | Glycerol and PEG | 2 |
| B29 | p-nitrophenyl carbonate | pentane | Glycerol and PEG | 2 |
| B30 | amine | — | Glycerol and PEG | 2 |
| B31 | thiol | butyrate | Lysine | 2 |
| B32 | thiol | glutarate | Lysine | 3 |
| B33 | aminoxy | PEG6 | Lysine | 3 |
| B34 | iodoacetamide | PEG6 | Lysine | 3 |
| B35 | Maleimide | C18-γGlu-20EG | Lysine | 3 |
| B36 | Maleimide | C18-γGlu-20EG | Lysine | 3 |
| B37 | Amine | EG2—NH— | Lysine | 3 |
| | | (CH2)5—COOH | | |
| B38 | N-hydroxysuccinimide | — | Lysine | 1 |
| B39 | N-hydroxysuccinimide | — | Lysine | 1 |
| B40 | Maleimide | — | | 1 |

B39 and B40 use desthiobiotin, an analog of biotin, as shown in Structure IV. 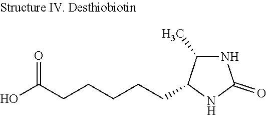
In some embodiments, biotin analog NHS-desthiobiotin as shown in Structure V, is used for conjugation.

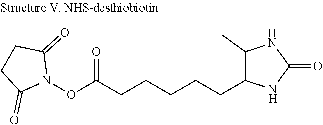 [SMILES: CC1NC(=O)NC1CCCCCC(=O)ON1C(=O)CCC1=O]

b. Lipidation

Lipidated peptides have an increased lipophilicity, an increased in vivo half-life (enabling once daily oral administration), and a reduced variability in pharmacokinetics at steady state. In some embodiments, an additional amino acid is added to the C-terminus of the GLP-1r agonist to enable the conjugation of one or more fatty acid molecules with increased linker stability. In preferred embodiments, the amino acid is either cysteine or lysine.

In some embodiments, the fatty acid moiety is conjugated to the GLP-1r agonist polypeptide via one or more additional cysteine or lysine residues added to the C-terminus of the polypeptide. In one embodiment, the fatty acid moiety is conjugated to the GLP-1r agonist polypeptide via one additional cysteine residue added to the C-terminus of the amino acid sequence represented by SEQ ID NO: 1, and the resulting peptide has the amino acid sequence of SEQ ID NO: 2. In another embodiment, the fatty acid moiety is conjugated to the GLP-1r agonist polypeptide via one additional lysine residue added to the C-terminus of the amino acid sequence represented by SEQ ID NO: 1, and the resulting peptide has the amino acid sequence of SEQ ID NO: 3. In yet another embodiment, one or more fatty acid moieties are conjugated to the GLP-1r agonist polypeptide via three additional lysine residues added to the C-terminus of the amino acid sequence of SEQ ID NO:1, and the resulting peptide has the amino acid sequence of SEQ ID NO:4. In some embodiments, the amino acid of the second position of SEQ ID NOs: 1-4 is substituted with 2-aminoisobutyric acid (Aib). In some embodiments, the fatty acid moiety is conjugated to the GLP-1r agonist polypeptide via one or more internal lysine residues, for example lysine at positions 12 and 27 of any of SEQ ID NOs: 1-8.

The first lipidated biopharmaceutical to obtain regulatory approval was insulin detemir in 2004. Insulin detemir, a basal insulin for the treatment of diabetes, includes desB30 human insulin conjugated to myristic acid (C14) through the Ne-amine of LysB29.

Current lipidated biopharmaceuticals have hydrophilic spacers, typically γGlu or OEG (8-amino-3,6-dioxaoctanoic acid), in between the lipid and peptide moieties to increase parameters such as albumin affinity, potency, water-solubility, and oligomerization. One example is liraglutide, a once-daily glucagon-like peptide 1 (GLP-1) analog marketed for treatment of diabetes and obesity. The liraglutide sequence is identical to that of native GLP-1 except for a Lys34Arg substitution, which enables selective palmitoylation through the Nε of Lys26 via a γGlu spacer (Lau J.; et al., J. Med. Chem. 2015, 58 (18), 7370-7380). Because of albumin binding and slow absorption, liraglutide has a significantly extended half-life (11-15 h, s.c.) compared to native GLP-1 (1-1.5 h, s.c.). From the dietary fatty acids used in insulin detemir and liraglutide, the preferred fatty acid for lipidation has advanced to the nondietary dicarboxylic fatty acids used in insulin degludec, a once-daily basal insulin, and semaglutide, a once-weekly GLP-1 analog. Insulin degludec is lipidated at LysB29 with a γGlu-spaced palmitic diacid. The peptide backbone of Semaglutide is similar to liraglutide's, except for a substitution of Alanine8 to 2-aminoisobutyric acid (Aib), which reduces degradation by dipeptidyl peptidase IV (DPP-4) (Lau, J. et al., Journal of Medicinal Chemistry (2015), 58 (18), 7370-7380). Semaglutide is lipidated at Lys26 with an octadecanoic diacid through a spacer including γGlu and two OEG units, which elicits an albumin affinity 5.6-fold larger than liraglutide's. The high albumin affinity as well as the DPP-4 resistance gives Semaglutide a half-life of approximately 1 week in humans (s.c.) (van Witteloostuijn, S. B.; Pedersen, S. L.; Jensen, K. J. ChemMedChem 2016, 11, 1-23). Impressively, this prolonged half-life is obtained without decreasing the GLP-1 receptor potency compared to the native ligand. Recently, lipidation has also been shown as a viable strategy for half-life extension of larger proteins as demonstrated by somapacitan, a once-weekly human growth hormone. The lipidation of somapacitan includes a significantly longer spacer region and a noncarboxylic fatty acid with a tetrazole headgroup.

Thus, in some embodiments, the long-acting GLP-1r agonist is conjugated to one or more of fatty acid chains, preferably with one or more hydrophilic spacers, such as γGlu or OEG (8-amino-3,6-dioxaoctanoic acid), in between the lipid and peptide. Exemplary fatty acids can include dietary fatty acids such as those used in insulin detemir and liraglutide, and the preferred fatty acids for lipidation are non-dietary dicarboxylic fatty acids such as those used in insulin degludec and semaglutide.

Exemplary fatty acid derivatives are shown in Table 4, below.

TABLE 4
Examples of fatty acid derivatives

| Fatty acid | |
|---|---|
| No. | Structure |
| F1 | C16-NHS |
| F2 | C16-MAL |
| F3 | C18-NHS |
| F4 | C18-MAL |
| F5 | C16-γGlu-NHS |
| F6 | C16-γGlu-MAL |
| F7 | C18-γGlu-NHS |
| F8 | C18-Glu-MAL |
| F9 | C18-γGlu-OEG-NHS |
| F10 | C18-γGlu-OEG-MAL |
| F11 | C18-γGlu-2OEG-NHS |
| F12 | C18-γGlu-2OEG-MAL |
| F13 | C20-γGlu-2OEG-NHS |
| F14 | C20-γGlu-2OEG-MAL |
| F15 | C18-γGlu-2OEG-TFP |
| F16 | C18-γGlu-2OEG-NPC |
| F17 | C20-γGlu-2OEG-NPC |

### 3. Exemplary Modified GLP-1r Agonists

In preferred embodiments, the long-acting GLP-1r agonist is lipidated and/or biotinylated exenatide. In particular embodiments, the long-acting GLP-1r agonist has the amino acid sequence of any one of SEQ ID NOs: 9-35 as shown in Table 5.

TABLE 5
Modifications to GLP-1r agonists

| Peptide | | Biotin deriv. | | Fatty acid deriv. | | |
|---|---|---|---|---|---|---|
| Peptide | | Biotin | Modification | Fatty acid | Modification | |
| No. | Name | No. | site | No. | site | SEQ ID NO |
| 12 | EX4C | B1 | C40 | — | — | SEQ ID NO: 9 |
| 12 | EX4C | B2 | C40 | — | — | SEQ ID NO: 10 |
| 12 | EX4C | B3 | C40 | — | — | SEQ ID NO: 11 |
| 5 | EX4 | B4 | K27 | — | — | SEQ ID NO: 12 |
| 12 | EX4C | B5 | C40 | — | — | SEQ ID NO: 13 |
| 12 | EX4C | B3 | C40 | F1 | K27 | SEQ ID NO: 14 |
| 12 | EX4C | B38 | K12, K27 | F2 | C40 | SEQ ID NO: 15 |
| 12 | EX4C | B3 | C40 | F11 | K27 | SEQ ID NO: 16 |
| 12 | EX4C | B38 | K12, K27 | F12 | C40 | SEQ ID NO: 17 |
| 12 | EX4C | B35 | C40 | — | — | SEQ ID NO: 18 |
| 12 | EX4C | B36 | C40 | — | — | SEQ ID NO: 19 |
| 18 | EX4K | B37 | K40 | — | — | SEQ ID NO: 20 |
| 19 | EX4KKK | B | K39, K40, | — | — | SEQ ID NO: 21 |
| | | | K41 | | | |
| 19 | EX4KKK | B | K39, K40, | F11 | K27 | SEQ ID NO: 22 |
| | | | K41 | | | |
| 19 | EX4KKK | B | K39, K40, | F11 | K12 | SEQ ID NO: 23 |
| | | | K41 | | | |
| 20 | EX4K(Aib) | B37 | K40 | — | — | SEQ ID NO: 24 |
| 21 | EX4KKK(Aib) | B | K39, K40, | — | — | SEQ ID NO: 25 |
| | | | K41 | | | |
| 21 | EX4KKK(Aib) | B | K39, K40, | F11 | K27 | SEQ ID NO: 26 |
| | | | K41 | | | |
| 21 | EX4KKK(Aib) | B | K39, K40, | F11 | K12 | SEQ ID NO: 27 |
| | | | K41 | | | |
| 20 | EX4K(Aib) | B37 | K40 | F11 | K27 | SEQ ID NO: 28 |
| 20 | EX4K(Aib) | B38 | K12, K27 | F11 | K40 | SEQ ID NO: 29 |
| 38 | EX01C | B38 | K12, K27 | F12 | C40 | SEQ ID NO: 30 |
| 39 | EX02C | B38 | K12, K27 | F12 | C40 | SEQ ID NO: 31 |
| 5 | EX4 | B38 | K12, K27 | — | — | SEQ ID NO: 32 |
| 12 | EX4C | — | — | F1 | C40 | SEQ ID NO: 33 |
| 12 | EX4C | — | — | F12 | C40 | SEQ ID NO: 34 |
| 12 | EX4C | B39 | K12, K27 | F12 | C40 | SEQ ID NO: 35 |

### 4. Other GLP-1r Agonists

In some embodiments, compositions include one or more GLP-1r agonists that is not exenatide, or an analog or derivative thereof. Exemplary GLP-1r agonists include GLP-1r agonists approved for treatment of type 2 diabetes, like Victoza (liraglutide), and Ozempic (semaglutide). Therefore, in some embodiments, one or more GLP-1r agonists are liraglutide or semaglutide.

### Dulaglutide

In some embodiments, the GLP-1r agonist suitable for biotinylation and/or lipidation is an Fc-fusion GLP-1 (e.g., dulaglutide), or an analog or derivative thereof. Dulaglutide is a glucagon-like peptide 1 receptor agonist (GLP-1 agonist) for the treatment of type 2 diabetes that can be used once weekly. Dulaglutide includes GLP-1 (7-37) covalently linked to an Fc fragment of human IgG₄, thereby protecting the GLP-1 moiety from inactivation by dipeptidyl peptidase 4.

GLP-1 is a hormone that is involved in the normalization of the level of glucose in blood (glycemia). GLP-1 is normally secreted by L cells of the gastrointestinal mucosa in response to a meal. Dulaglutide binds to glucagon-like peptide 1 receptors, slowing gastric emptying and increasing insulin secretion by pancreatic Beta cells. Dulaglutide simultaneously reduces the elevated glucagon secretion by inhibiting alpha cells of the pancreas.

### Albiglutide

In some embodiments, the GLP-1r agonist suitable for biotinylation and/or lipidation is an albumin-fusion GLP-1 (e.g., albiglutide), or an analog or derivative thereof. Albiglutide is a glucagon-like peptide-1 agonist (GLP-1 agonist) drug used for treatment of type 2 diabetes. It is a dipeptidyl peptidase-4-resistant glucagon-like peptide-1 dimer fused to human albumin. Albiglutide has a half-life of four to seven days.

In some embodiments, the GLP-1 agonist suitable for biotinylation and/or lipidation is a PEGylated GLP-1 analog or derivative thereof (e.g., NLY01, a pegylated exendin-4 analogue of GLP-1r agonist).

Other examples of therapeutic active agents include DPPIV inhibitors, which increase native GLP-1R levels by inhibiting the protease dipeptidyle peptidase IV, which is responsible for rapid inactivation of GLP-1 in circulation. Medicines in the DPP-4 inhibitor class include JANUVIA® (sitagliptin), ONGLYZA® (saxagliptin), TRADJENTA® (linagliptin), and NESINA® (alogliptin). Each of these is also available as a combination product with other anti-diabetic drugs such as metformin. Enhancement of native GLP-1 levels through inhibition of DPPIV is another means of targeting the GLP-1R to reduce microglial activation. Therefore, in some embodiments, the GLP-1r agonist is a DPPIV inhibitor, for example, sitagliptin, saxagliptin, linagliptin, or alogliptin.

### B. Additional Active Agents

In some embodiments the long acting GLP-1r agonists are administered to a subject together with one or more additional active agents, particularly one or more antiviral or antimicrobial agents, or additional agents to prevent or treat one or more symptoms of a neurological or neurodegenerative disease or disorder. Suitable therapeutic, diagnostic, and/or prophylactic agents can be a biomolecule, such as peptides, proteins, carbohydrates, nucleotides or oligonucleotides, or a small molecule agent (e.g., molecular weight less than 2000 amu, preferably less than 1500 amu), including organic, inorganic, and organometallic agents.

### 1. Therapeutic and Prophylactic Agents

In some embodiments, the long acting GLP-1r agonists are administered to a subject together with one or more additional therapeutic, prophylactic, or prognostic agents. Representative therapeutic agents include, but are not limited to, neuroprotective agents, anti-inflammatory agents, antioxidants, anti-infectious agents, and combinations thereof.

In one embodiment, the additional agent is a steroid. Suitable steroids include biologically active forms of vitamin D3 and D2, such as those described in U.S. Pat. Nos. 4,897,388 and 5,939,407. The steroids may be co-administered to further aid in neurogenic stimulation or induction and/or prevention of neural loss. Estrogen and estrogen related molecules such as allopregnanolone can be co-administered with the neuro-enhancing agents to enhance neuroprotection, as described in Brinton (2001) Learning and Memory 8 (3): 121-133.

Other neuroactive steroids, such as various forms of dehydroepi-androsterone (DHEA), as described in U.S. Pat. No. 6,552,010, can also be co-administered to further aid in neurogenic stimulation, induction and/or prevention of neural loss. Other agents that cause neural growth and outgrowth of neural networks, such as Nerve Growth Factor (NGF) and Brain-derived Neurotrophic Factor (BDNF), can be administered either simultaneously with, before or after the administration of THP. Additionally, inhibitors of neural apoptosis, such as inhibitors of calpains and caspases and other cell death mechanisms, such as necrosis, can be co-administered with the neuro-enhancing agents to further prevent neural loss associated with certain neurological diseases and neurological defects.

### C. Pharmaceutical Formulations

In some embodiments, the long-acting GLP-1r agonists are formulated with one or more pharmaceutical excipients, additives, or fillers. For example, in some embodiments, the long-acting GLP-1r agonists are formulated into pharmaceutical formulations for administration to a subject. Compositions including long-acting GLP-1r agonists may be formulated in a conventional manner using one or more physiologically acceptable carriers including excipients and auxiliaries which facilitate processing of the active compounds into preparations which can be used pharmaceutically.

Proper formulation is dependent upon the route of administration chosen. In preferred embodiments, the compositions are formulated for parenteral delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain long-acting GLP-1r agonists in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

### 1. Dosage Units

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rats, rabbits, dogs, or pigs. The animal model is also used to achieve a desirable concentration range and route of administration. Such information should then be useful to determine useful doses and routes for administration in humans.

### 2. Formulations for Administration

In some embodiments, the compositions of long-acting GLP-1r agonists are formulated into a pharmaceutically acceptable formulation for administration via a specific route. In some embodiments, the compositions are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in some embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous (IV) or subcutaneous injection) and enteral routes of administration are described.

a. Enteral Administration

In some embodiments, long-acting GLP-1r agonists are administered orally. For oral administration, suitable formulations include tablets, pellets, hard/soft capsules, liquids, suspensions, emulsifiers, syrups, granules, elixirs, troches, etc., and these formulations can include diluents (for example, lactose, dextrose, sucrose, mannitol, sorbitol, cellulose and/or glycine), slip modifiers (for example, silica, talc, stearate and its magnesium or calcium salt and/or polyethylene glycol) in addition to the active ingredient. Tablets may also include binders such as magnesium aluminum silicate, starch paste, gelatin, methyl cellulose, sodium carboxymethyl cellulose and/or polyvinyl pyrrolidine, and may include disintegrating agents such as starch, agar, alginic acid or sodium salt thereof or boiling mixture and/or absorbents, coloring agents, flavoring agents and sweetening agents if needed.

In preferred embodiments, solid formulations include the long-acting GLP-1r agonists (API), permeation enhancers (PE), stability enhancer (SE), binder, disintegrant, glidant, and lubricant. Exemplary permeation enhancers include bile acid, cholic acid, deoxycholic acid, glycocholic acid, glycochonodeoxycholic acid, taurochenodeoxycholic acid, taurocholic acid, chenodeoxycholic acid, ursodeoxycholic acid, lithocholic acid, Labrasol (Caprylocaproyl Polyoxyl-8 glycerides), SNAC (sodium N-(8-[2-hydroxybenzoyl]amino) caprylate, and their salt forms. An exemplary stability enhancer is propyl gallate. In some embodiments, for in vitro and/or in vivo studies, the ratio of API to (PE+SE) is between about 1:0.01 w/w and about 1:1,000 w/w, inclusive. In some embodiments, the ratio of PE to SE is between 1:0.01 w/w and about 1:8 w/w, inclusive. In preferred embodiments, solid formulations such as in tablet form include between about 1 mg and about 50 mg of API, between about 1 mg and about 1,000 mg of bile acid, and between about 1 mg and about 1,001 mg of propyl gallate. Exemplary diluents or fillers include lactose, starch, microcrystalline cellulose, and mannitol. Exemplary lubricants include magnesium stearate and sodium stearyl fumarate.

b. Parenteral Administration

In some embodiments, long-acting GLP-1r agonists are formulated into a pharmaceutically acceptable formulation for parenteral administration. The phrases “parenteral administration” and “administered parenterally” are art-recognized terms, and include modes of administration other than enteral and topical administration, such as injections, and include without limitation intravenous (i.v.), intramuscular (i.m.), intraperitoneal (i.p.), subcutaneous (s.c.) injection and infusion. The long-acting GLP-1r agonists can be administered parenterally, for example, by intravenous, intraperitoneal, or subcutaneous routes.

For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The long-acting GLP-1r agonists can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

In preferred embodiments, aqueous formulation including long-acting GLP-1r agonists (e.g., DD02S), a buffer, a tonicity agent, water, preservative, and stabilizing agent. Typically, the long-acting GLP-1r agonists are in a concentration between about 0.1 mg/mL and about 10 mg/mL, inclusive. An exemplary buffer is phosphate in a concentration between about 5 mM and about 45 mM, inclusive; at a pH between about 5.5 and about 9.0, inclusive. Exemplary tonicity agents include potassium chloride, sodium chloride, and mannitol.

### III. Methods of Making

### A. Methods of Making Long-Acting GLP-1r Agonists

Long-acting GLP-1r agonists can be prepared via a variety of chemical reaction steps. Typically, methods for making Long-acting GLP-1r agonists include biotinylation and/or lipidation.

### 1. Biotinylation and Lipidation

Biotin modifications to exendin derivatives have been previously described, for example, in International Publication Nos. WO2009107900A1, WO2020242268A1, and WO2021107519A1, the contents of which are incorporated by reference herein in their entirety.

In some embodiments, biotinylation and lipidation are carried out simultaneously. In one embodiment, the methods include dissolving the peptide, C16-MAL (F2) or C18-γGlu-20EG-MAL (F12), and biotin-N-hydroxysuccinimide ester (B1-NHS, B38) in DMSO containing 0.3% TEA (v/v) solution. In one embodiment, the peptide and F2 or F12 are mixed at a volume ratio of 1:1. The concentration of peptide is 10 mg/mL and the molar ratio is 1:2 at this step. In one embodiment, the mixture is reacted at 25° C. for 10 min with gently shaking. In another embodiment, B1-NHS is added at a volume ratio of 1:1. The concentration of peptide is 5 mg/mL and the molar ratio is 1:2 or 1:3. In one embodiment, the mixture is reacted at 25° C. for 90 min with gently shaking.

In another embodiment, the methods include dissolving the peptide (e.g., SEQ ID NO: 11) and C16-NHS (F1) or C18-γGlu-20EG-TFP (F15) in DMSO containing 0.3% TEA (v/v) solution; and mixing each solution at a volume ratio of 1:1. In one embodiment, the concentration of peptide is 5 mg/mL and the molar ratio is 1:2. The mixture is reacted at 25° C. for 30 min with gently shaking.

In another embodiment, the methods include dissolving the peptide, B35 or B36 in DMSO containing 0.3% TEA (v/v) solution; and mixing each solution at a volume ratio of 1:1. In one embodiment, the concentration of peptide is 5 mg/mL and the molar ratio is 1:2. The mixture is then reacted at 25° C. for 10 min with gently shaking.

In one embodiment, lipidated and biotinylated peptides are purified by Prep-LC and the eluate can be collected in individual fractions. In one embodiment, the ACN contained in the fractionated solution is evaporated using the centrifugal evaporator at 45° C. for 40 min. The solvent can be changed to water by ultrafiltration. The purified samples can be analyzed by reversed phase-HPLC for purity check. In one embodiment, the samples are lyophilized at −88° C. for 18 hr and then stored at −20° C.

### 2. Lipidation

In one embodiment, the methods include dissolving the peptide and C16-NHS (F1) or C18-γGlu-20EG-MAL (F12) in dimethyl sulfoxide (DMSO) containing 0.3% triethylamine (TEA) (v/v) solution; and mixing each solution at a volume ratio of 1:1. In one embodiment, the concentration of peptide is 5 mg/mL, and the molar ratio is 1:2. The mixture is then reacted at 25° C. for 10 min with gently shaking. In one embodiment, lipidated peptides are purified by Prep-LC and the eluate is collected in individual fractions. In one embodiment, the ACN contained in the fractionated solution is evaporated using the centrifugal evaporator at 45° C. for 40 min. The solvent can be changed to water by ultrafiltration. The purified samples can then be analyzed by reversed phase-HPLC for purity check. In one embodiment, the samples are lyophilized at −88° C. for 18 hr and then stored at −20° C.

### IV. Methods of Use

Methods of using the glucagon like peptide 1 receptor agonists (GLP-1r agonists) are described. Typically, the GLP-1r agonists are biotinylated and/or lipidated GLP-1r agonists (long-acting GLP-1r agonists). In preferred embodiments, the GLP-1r agonists cross the blood brain barrier (BBB) and selectively target activated microglia and/or reactive astrocytes.

### A. Methods of Treatment

The disclosed GLP-1r agonists cross impaired or damaged BBB and target activated microglia and/or reactive astrocytes in the brain to reduce or prevent neurological diseases or disorders in a subject.

Methods of using the disclosed GLP-1r agonists for treating or preventing neurological diseases or disorders are described. Methods for blocking or reducing microglial activation and/or reactive astrocytes in one or more neurological or neurodegenerative diseases with minimal off-target toxicity are also described. In preferred embodiments, the disclosed GLP-1r agonists are administered in an amount and with a dosing regimen effective to prevent, inhibit, or reduce one or more symptoms associated with one or more neurological diseases or disorders in the subject. The disclosed GLP-1r agonists are administered to a subject in one or multiple doses, at one or multiple time points following an initial dose. The amount of composition administered to the subject is selected to deliver an effective amount to safely reduce, prevent, or otherwise alleviate one or more clinical or molecular symptoms of the disease or disorder to be treated compared to a control, for example, a subject treated with a short-acting GLP-1r agonist (exenatide and liraglutide). The compositions and methods are also suitable for prophylactic use.

### 1. Traversing the Blood Brain Barrier (BBB)

The long-acting GLP-1r agonists traverse the blood brain barrier without the use of targeting or trafficking moieties. In preferred embodiments, the long-acting GLP-1r agonists disclosed here provide better permeation than semaglutide or NLY01. In the case of biotinylated GLP-1r agonists, SMVT mediate transport is involved.

The blood-brain barrier (BBB) is a highly selective permeability barrier that separates the circulating blood from the brain extracellular fluid (BECF) in the central nervous system (CNS). The blood-brain barrier is formed by brain endothelial cells, which are connected by tight junctions with an extremely high electrical resistivity. Astrocytes are necessary to create the blood-brain barrier. The blood-brain barrier allows the passage of lipid-soluble molecules, water, and some gases by passive diffusion, as well as the selective transport of molecules such as amino acids and glucose which are crucial to neural function. The blood-brain barrier occurs along all brain capillaries and includes tight junctions around the capillaries that do not exist in normal circulation. Endothelial cells restrict the diffusion of microscopic objects (e.g., bacteria) and large or hydrophilic molecules into the cerebrospinal fluid (CSF), while allowing the diffusion of small hydrophobic molecules (e.g., O₂, CO₂, hormones). Cells of the barrier actively transport metabolic products such as glucose across the barrier with specific proteins. This “barrier” results from the selectivity of the tight junctions between endothelial cells in CNS vessels that restricts the passage of solutes. At the interface between blood and the brain, endothelial cells are stitched together by these tight junctions, which are composed of smaller subunits, frequently biochemical dimers, that are transmembrane proteins such as occludin, claudins, junctional adhesion molecule (JAM), or ESAM, for example. Each of these transmembrane proteins is anchored into the endothelial cells by another protein complex that includes zo-1 and associated proteins.

The blood-brain barrier is formed by the brain capillary endothelium and excludes from the brain approximately 100% of large-molecule neurotherapeutics and more than 98% of all small-molecule drugs. Overcoming the difficulty of delivering therapeutic agents to specific regions of the brain presents a major challenge to treatment of most brain disorders. In its neuroprotective role, the blood-brain barrier functions to hinder the delivery of many potentially important diagnostic and therapeutic agents to the brain. Therapeutic molecules and antibodies that might otherwise be effective in diagnosis and therapy do not cross the BBB in adequate amounts. Mechanisms for drug targeting in the brain involve going either “through” or “behind” the BBB. Modalities for drug delivery/Dosage form through the BBB entail its disruption by osmotic means; biochemically by the use of vasoactive substances such as bradykinin; or even by localized exposure to high-intensity focused ultrasound (HIFU). Other methods used to get through the BBB may entail the use of endogenous transport systems, including carrier-mediated transporters such as glucose and amino acid carriers; receptor-mediated transcytosis for insulin or transferrin; and the blocking of active efflux transporters such as p-glycoprotein. However, vectors targeting BBB transporters, such as the transferrin receptor, have been found to remain entrapped in brain endothelial cells of capillaries, instead of being ferried across the BBB into the cerebral parenchyma. Methods for drug delivery behind the BBB include intracerebral implantation (e.g., using needles) and convection-enhanced distribution. Additionally, mannitol can be used in bypassing the BBB.

### 2. Modulation of Activated Microglia

Microglia are a type of neuroglia (glial cell) located throughout the brain and spinal cord. Microglia account for 10-15% of all cells found within the brain. As the resident macrophage cells, they act as the first and main form of active immune defense in the central nervous system (CNS). Microglia play a key role after CNS injury and can have both protective and deleterious effects based on the timing and type of insult. Changes in microglial function also affect normal neuronal development and synaptic pruning. Microglia undergo a pronounced change in morphology from ramified to an amoeboid structure and proliferate after injury. The resulting neuroinflammation disrupts the blood-brain-barrier at the injured site and cause acute and chronic neuronal and oligodendrocyte death. Hence, targeting pro-inflammatory microglia should be a potent and effective therapeutic strategy.

The long-acting GLP-1r agonists selectively target and block microglia and astrocytes activation and the release of inflammatory and neurotoxic molecules from activated resident innate immune cells; thus prevent, stop and/or ameliorate the progression of neurodegenerative diseases. It was discovered that microglia activated by abnormally aggregated proteins upregulate GLP-1r and a long-acting GLP-1r agonist bound to activated microglia significantly inhibit the release of toxic molecules including TNF-α, IL-1α, IL-1β, IL-6, and C1q and protect neurons. Through GLP-1r internalization assay, it was discovered that a long-acting GLP-1r agonist demonstrates slow internalization of GLP-1r, reduces the rate of GLP-1r recycling compared to that of short-acting GLP-1r agonists (exenatide and liraglutide), thus can continuously activate GLP-1r and induce GLP-1r signaling in the brain. Patients treated with a short-acting GLP-1r agonist would experience “off time” that will mar the therapeutic effect during a chronic treatment. In contrast, the long-acting GLP-1r agonists has the ability to penetrate BBB and activate GLP-1r in the brain in a continuous fashion without “off time” and without off-target toxicity.

In some embodiments, the long-acting GLP-1r agonists function to ameliorate neurological diseases and disorders by reducing or inhibiting the activation of microglia and/or astrocytes. In response to neurodegeneration and the accumulation of abnormally aggregated proteins, such as «-synuclein and β-amyloid, resting microglia become an activated state and release various cytokines and neurotoxic molecules including TNF-α, IL-1α, IL-1β, IL-6, and C1q that drive their proliferation and activate astrocytes. Consequently, such inflammatory mediators released from activated microglia or reactive astrocytes, induced by activated microglia, causes neuronal damage, and contribute to the progression of neurodegenerative diseases. Therefore, activated microglia can be described as major upstream bad agents in neurodegenerative diseases. Inhibition of microglia activation without off-target toxicity is effective to prevent, stop and/or reverse the neurodegeneration process. However, the lack of translational methods to specifically target microglia activation hampered this strategy.

Preferably, the compositions protect against alpha-synuclein associated loss of dopaminergic neurons. In one embodiment, the compositions protect against amyloid-beta and/or tau toxicity in Alzheimer's disease neurons. For example, the compositions protect against amyloid plaques and tau-associated loss of neurons. In another embodiment, the compositions improve motor and cognitive as well as memory skills in a subject relative to a control. In a further embodiment, the compositions protect synapses and/or synaptic functions, enhance neurogenesis, reduce apoptosis, protect neurons from oxidative stress, reduce plaque formation, and prevent chronic inflammatory response in a subject relative to a control.

### B. Conditions to be Treated

The compositions are suitable for treating one or more diseases, conditions, and injuries in the brain, and the nervous system, particularly those associated with pathological activation of microglia and/or astrocytes. In preferred embodiments, the compositions are administered in an amount effective to treat microglial-mediated pathology in the subject in need thereof without any associated toxicity.

In some embodiments, the subject to be treated is a human. In some embodiments, the subject to be treated is a child, or an infant. All the methods can include the step of identifying and selecting a subject in need of treatment, or a subject who would benefit from administration with the described compositions.

### 1. Neurological and Neurodegenerative Diseases

The compositions and formulations thereof can be used to treat one or more neurological and neurodegenerative diseases. The compositions and methods are particularly suited for treating one or more neurological or neurodegenerative diseases associated with activation of microglia and/or astrocytes. In some embodiments, the disease or disorder is selected from, but not limited to, neurological disorders (e.g., Alzheimer's disease (AD), Parkinson's disease (PD)). In one embodiment, the compositions are used to treat Alzheimer's Disease (AD) or Parkinson's disease.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target. Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia. In preferred embodiments, the disease or disorder is Alzheimer's Disease (AD) or Parkinson's disease.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the compositions. Following this initial assessment, treatment methods for the administration of the compositions are initiated and continued for various time intervals. At a selected time interval subsequent to the initial assessment of the neurological defect impairment, the same assessment or evaluation test(s) is again used to reassess changes or improvements in selected neurological criteria.

a. Alzheimer's Disease

Alzheimer's disease (AD) accounts for 60% to 70% of cases of dementia. It is a chronic neurodegenerative disease that often starts slowly, but progressively worsens over time. The most common early symptom is short-term memory loss. As the disease advances, symptoms include problems with language, mood swings, loss of motivation, disorientation, behavioral issues, and poorly managed self-care. Gradually, bodily functions are lost, ultimately leading to death. Although the speed of progression can vary, the average life expectancy following diagnosis is three to nine years. The cause of Alzheimer's disease is poorly understood. About 70% of the risk is believed to be genetic with many genes involved. Other risk factors include a history of head injuries, hypertension, or depression. The disease process is associated with plaques and tangles in the brain.

Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Alzheimer's disease has been hypothesized to be a protein misfolding disease (proteopathy), caused by accumulation of abnormally folded A-beta and tau proteins in the brain. Plaques are made up of small peptides, 39-43 amino acids in length, called beta-amyloid (also written as A-beta or AB). Beta-amyloid is a fragment from a larger protein called amyloid precursor protein (APP), a transmembrane protein that penetrates through the neuron's membrane. APP is critical to neuron growth, survival, and post-injury repair. In Alzheimer's disease, an unknown process causes APP to be divided into smaller fragments by enzymes through proteolysis. One of these fragments gives rise to fibrils of beta-amyloid, which form clumps that deposit outside neurons in dense formations known as senile plaques.

A probable diagnosis is based on the history of the illness and cognitive testing with medical imaging and blood tests to rule out other possible causes. Initial symptoms are often mistaken for normal ageing. Examination of brain tissue is needed for a definite diagnosis. Alzheimer's disease is diagnosed through a complete medical assessment. There is no one clinical test that can determine whether a person has Alzheimer's. Usually, several tests are performed to rule out any other cause of dementia. The only definitive method of diagnosis is examination of brain tissue obtained from a biopsy or autopsy. Tests (such as blood tests and brain imaging) are used to rule out other causes of dementia-like symptoms. Laboratory tests and screening include complete blood cell count; electrolyte panel; screening metabolic panel; thyroid gland function tests; vitamin B-12 folate levels; tests for syphilis and, depending on history, for human immunodeficiency antibodies; urinalysis; electrocardiogram (ECG); chest X-ray; computerized tomography (CT) head scan; and an electroencephalogram (EEG). A lumbar puncture may also be informative in the overall diagnosis.

There are no medications or supplements that decrease risk. No treatments stop or reverse its progression, though some may temporarily improve symptoms.

The compositions and formulations are suitable for reducing or preventing one or more pathological processes associated with the development and progression of neurological diseases such as Alzheimer's disease. Thus, methods for treatment, reduction, and prevention of the pathological processes associated with Alzheimer's disease include administering the compositions in an amount and dosing regimen effective to reduce microglial activation, total Aβ₄₂ and plaque burden, tau phosphorylation/propagation, and/or improved cognition in a learning task, such as a fear-conditioned learning task, in an individual suffering from Alzheimer's disease are provided. Methods for reducing, preventing, or reversing the learning and/or memory deficits in an individual suffering from Alzheimer's disease or dementia are provided. The methods include administering an effective amount of a composition including one or more long-acting GLP-1r agonists to a subject in need thereof. In preferred embodiments, the methods include administering an effective amount of a composition including one or more long-acting GLP-1r agonists having amino acid sequence of any one of SEQ ID NOs: 1-35, or pharmaceutically acceptable salt thereof to the subject.

In some embodiments, the compositions are administered in an amount and dosing regimen effective to induce neuro-enhancement in a subject in need thereof. Neuro-enhancement resulting from the administration of the compositions includes the stimulation or induction of neural mitosis leading to the generation of new neurons, i.e., exhibiting a neurogenic effect, prevention or retardation of neural loss, including a decrease in the rate of neural loss, i.e., exhibiting a neuroprotective effect, or one or more of these modes of action. The term “neuroprotective effect” is intended to include prevention, retardation, and/or termination of deterioration, impairment, or death of an individual's neurons, neurites, and neural networks. Administration of the compositions leads to an improvement, or enhancement, of neurological function in an individual with a neurological disease, neurological injury, or age-related neuronal decline or impairment.

Neural deterioration can be the result of any condition which compromises neural function which is likely to lead to neural loss. Neural function can be compromised by, for example, altered biochemistry, physiology, or anatomy of a neuron, including its neurite. Deterioration of a neuron may include membrane, dendritic, or synaptic changes, which are detrimental to normal neuronal functioning. The cause of the neuron deterioration, impairment, and/or death may be unknown. Alternatively, it may be the result of age-, injury- and/or disease-related neurological changes that occur in the nervous system of an individual.

In Alzheimer's patients, neural loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory.

Neural loss through disease, age-related decline or physical insult leads to neurological disease and impairment. The compositions can counteract the deleterious effects of neural loss by promoting development of new neurons, new neurites and/or neural connections, resulting in the neuroprotection of existing neural cells, neurites and/or neural connections, or one or more these processes. Thus, the neuro-enhancing properties of the compositions provide an effective strategy to generally reverse the neural loss associated with degenerative diseases, aging and physical injury or trauma.

Administration of the compositions to an individual who is undergoing or has undergone neural loss, as a result of Alzheimer's disease, reduces any one or more of the symptoms of Alzheimer's disease, or associated cognitive disorders, including dementia. Clinical symptoms of AD or dementia that can be treated, reduced, or prevented include clinical symptoms of mild AD, moderate AD, and/or severe AD or dementia.

In mild Alzheimer's disease, a person may seem to be healthy but has more and more trouble making sense of the world around him or her. The realization that something is wrong often comes gradually to the person and their family. Exemplary symptoms of mild Alzheimer's disease/mild dementia include, but are not limited to, memory loss; poor judgment leading to bad decisions; loss of spontaneity and sense of initiative; taking longer to complete normal daily tasks; repeating questions; trouble handling money and paying bills; wandering and getting lost; losing things or misplacing them in odd places; mood and personality changes, and increased anxiety and/or aggression.

Symptoms of moderate Alzheimer's disease/moderate dementia include, but are not limited to forgetfulness; increased memory loss and confusion; inability to learn new things; difficulty with language and problems with reading, writing, and working with numbers; difficulty organizing thoughts and thinking logically; shortened attention span; problems coping with new situations; difficulty carrying out multistep tasks, such as getting dressed; problems recognizing family and friends; hallucinations, delusions, and paranoia; impulsive behavior such as undressing at inappropriate times or places or using vulgar language; inappropriate outbursts of anger; restlessness, agitation, anxiety, tearfulness, wandering (especially in the late afternoon or evening); repetitive statements or movement, occasional muscle twitches.

Symptoms of severe Alzheimer's disease/severe dementia include, but are not limited to inability to communicate; weight loss; seizures; skin infections; difficulty swallowing; groaning, moaning, or grunting; increased sleeping; loss of bowel and bladder control.

Physiological symptoms of Alzheimer's disease include reduction in brain mass, for example, reduction in hippocampal volume. Therefore, in some embodiments, methods of administering the compositions increase the brain mass, and/or reduce or prevent the rate of decrease in brain mass of a subject; increase the hippocampal volume of the subject, reduce, or prevent the rate of decrease of hippocampal volume, as compared to an untreated control subject.

In further embodiments, methods of administering the compositions in an effective amount to reduce microglial activation, total Aβ₄₂ and plaque burden, tau phosphorylation, improved or ameliorate neurological defects or cognitive decline or impairment, and combinations thereof. A therapeutic effect is generally observed within about 12 to about 24 weeks of initiating administration, although the therapeutic effect may be observed in less than 12 weeks or greater than 24 weeks.

The individual is preferably an adult human, and more preferably, a human is over the age of 30, who has lost some amount of neurological function as a result of Alzheimer's disease or dementia. Generally, neural loss implies any neural loss at the cellular level, including loss in neurites, neural organization, or neural networks.

In other embodiments, the methods including selecting a subject who is likely to benefit from treatment with the compositions.

b. Parkinson's Disease

Parkinson's disease (PD, also known as idiopathic or primary parkinsonism, hypokinetic rigid syndrome (HRS), or paralysis agitans) is a degenerative disorder of the central nervous system mainly affecting the motor system. The motor symptoms of Parkinson's disease result from the death of dopamine-generating cells in the substantia nigra, a region of the midbrain. The causes of this cell death are poorly understood. Early in the course of the disease, the most obvious symptoms are movement-related; these include shaking, rigidity, slowness of movement and difficulty with walking and gait. Later, thinking and behavioral problems may arise, with dementia commonly occurring in the advanced stages of the disease, and depression is the most common psychiatric symptom. Other symptoms include sensory, sleep and emotional problems. Parkinson's disease is more common in older people, with most cases occurring after the age of 50; when it is seen in young adults, it is called young onset PD (YOPD).

The main motor symptoms are collectively called “parkinsonism,” or a “parkinsonian syndrome.” The disease can be either primary or secondary. Primary Parkinson's disease is referred to as idiopathic (having no known cause), although some atypical cases have a genetic origin, while secondary parkinsonism is due to known causes like toxins. The pathology of the disease is characterized by the accumulation of a protein into Lewy bodies in neurons, and insufficient formation and activity of dopamine in certain parts of the midbrain. Where the Lewy bodies are located is often related to the expression and degree of the symptoms of an individual. Diagnosis of typical cases is mainly based on symptoms, with tests such as neuroimaging being used for confirmation.

Diagnosis of Parkinson's disease involves a physician taking a medical history and performing a neurological examination. There is no lab test that will clearly identify the disease, but brain scans are sometimes used to rule out disorders that could give rise to similar symptoms. People may be given levodopa and resulting relief of motor impairment tends to confirm diagnosis. The finding of Lewy bodies in the midbrain on autopsy is usually considered proof that the person had Parkinson's disease. The progress of the illness over time may reveal it is not Parkinson's disease, and some authorities recommend that the diagnosis be periodically reviewed. Other causes that can secondarily produce a parkinsonian syndrome are Alzheimer's disease, multiple cerebral infarction and drug-induced parkinsonism. Parkinson plus syndromes such as progressive supranuclear palsy and multiple system atrophy must be ruled out. Anti-Parkinson's medications are typically less effective at controlling symptoms in Parkinson plus syndromes. Faster progression rates, early cognitive dysfunction or postural instability, minimal tremor or symmetry at onset may indicate a Parkinson plus disease rather than PD itself. Genetic forms are usually classified as PD, although the terms familial Parkinson's disease and familial parkinsonism are used for disease entities with an autosomal dominant or recessive pattern of inheritance.

The PD Society Brain Bank criteria require slowness of movement (bradykinesia) plus either rigidity, resting tremor, or postural instability. Other possible causes for these symptoms need to be ruled out prior to diagnosis with PD. Finally, three or more of the following features are required during onset or evolution: unilateral onset, tremor at rest, progression in time, asymmetry of motor symptoms, response to levodopa for at least five years, clinical course of at least ten years and appearance of dyskinesias induced by the intake of excessive levodopa. Accuracy of diagnostic criteria evaluated at autopsy is 75-90%, with specialists such as neurologists having the highest rates. Computed tomography (CT) and conventional magnetic resonance imaging (MRI) brain scans of people with PD usually appear normal. These techniques are nevertheless useful to rule out other diseases that can be secondary causes of parkinsonism, such as basal ganglia tumors, vascular pathology, and hydrocephalus. A specific technique of MRI, diffusion MRI, has been reported to be useful at discriminating between typical and atypical parkinsonism, although its exact diagnostic value is still under investigation. Dopaminergic function in the basal ganglia can be measured with different PET and SPECT radiotracers. Examples are ioflupane (1231) (trade name DaTSCAN) and iometopane (Dopascan) for SPECT or fluorodeoxyglucose (18F) and DTBZ for PET. A pattern of reduced dopaminergic activity in the basal ganglia can aid in diagnosing PD.

Treatments, typically the medications L-DOPA and dopamine agonists, improve the early symptoms of the disease. As the disease progresses and dopaminergic neurons continue to be lost, these drugs eventually become ineffective at treating the symptoms and at the same time produce a complication marked by involuntary writhing movements. Surgery and deep brain stimulation have been used to reduce motor symptoms as a last resort in severe cases where drugs are ineffective. Although dopamine replacement alleviates the symptomatic motor dysfunction, its effectiveness is reduced as the disease progresses, leading to unacceptable side effects such as severe motor fluctuations and dyskinesias. Furthermore, there is no therapy that will halt the progress of the disease (Lang, A. E. and A. M. Lozano, N Engl J Med, 1998. 339(15): p. 1044-53; Lang, A. E. and A. M. Lozano, N Engl J Med, 1998. 339(16): p. 1130-43). Moreover, this palliative therapeutic approach does not address the underlying mechanisms of the disease (Nagatsua, T. and M. Sawadab, Parkinsonism Relat Disord, 2009. 15 Suppl 1: p. S3-8).

The term parkinsonism is used for a motor syndrome whose main symptoms are tremor at rest, stiffness, slowing of movement and postural instability. Parkinsonian syndromes can be divided into four subtypes according to their origin: primary or idiopathic, secondary or acquired, hereditary parkinsonism, and Parkinson plus syndromes or multiple system degeneration. Usually classified as a movement disorder, PD also gives rise to several non-motor types of symptoms such as sensory deficits, cognitive difficulties, or sleep problems. Parkinson plus diseases are primary parkinsonisms which present additional features. They include multiple system atrophy, progressive supranuclear palsy, corticobasal degeneration and dementia with Lewy bodies.

In terms of pathophysiology, PD is considered a synucleinopathy due to an abnormal accumulation of alpha-synuclein protein in the brain in the form of Lewy bodies, as opposed to other diseases such as Alzheimer's disease where the brain accumulates tau protein in the form of neurofibrillary tangles. Nevertheless, there is clinical and pathological overlap between tauopathies and synucleinopathies. The most typical symptom of Alzheimer's disease, dementia, occurs in advanced stages of PD, while it is common to find neurofibrillary tangles in brains affected by PD. Dementia with Lewy bodies (DLB) is another synucleinopathy that has similarities with PD, and especially with the subset of PD cases with dementia. However, the relationship between PD and DLB is complex and still has to be clarified. They may represent parts of a continuum, or they may be separate diseases.

Mutations in specific genes have been conclusively shown to cause PD. These genes code for alpha-synuclein (SNCA), parkin (PRKN), leucine-rich repeat kinase 2 (LRRK2 or dardarin), PTEN-induced putative kinase 1 (PINK1), DJ-1 and ATP13A2. In most cases, people with these mutations will develop PD. With the exception of LRRK2, however, they account for only a small minority of cases of PD. The most extensively studied PD-related genes are SNCA and LRRK2. Mutations in genes including SNCA, LRRK2 and glucocerebrosidase (GBA) have been found to be risk factors for sporadic PD. Mutations in GBA are known to cause Gaucher's disease. Genome-wide association studies, which search for mutated alleles with low penetrance in sporadic cases, have now yielded many positive results.

The role of the SNCA gene is important in PD because the alpha-synuclein protein is the main component of Lewy bodies. The histopathology (microscopic anatomy) of the substantia nigra and several other brain regions shows neuronal loss and Lewy bodies in many of the remaining nerve cells. Neuronal loss is accompanied by death of astrocytes (star-shaped glial cells) and activation of the microglia (another type of glial cell). Lewy bodies are a key pathological feature of PD.

The compositions and formulations are suitable for reducing or preventing one or more pathological processes associated with the development and progression of PD. Thus, methods for treatment, reduction, and prevention of the pathological processes associated with PD include administering the compositions in an amount and dosing regimen effective to reduce microglial activation, abnormal accumulation of alpha-synuclein protein, neurofibrillary tangles in brains, and/or improved shaking, rigidity, slowness of movement and difficulty with walking, in an individual suffering from PD are provided. Methods for reducing, preventing, or reversing the motor dysfunction in an individual suffering from PD are provided. The methods include administering an effective amount of a composition including one or more long-acting GLP-1r agonists to a subject in need thereof. In preferred embodiments, the methods include administering an effective amount of a composition including one or more long-acting GLP-1r agonists having amino acid sequence of any one of SEQ ID NOs: 1-35, or pharmaceutically acceptable salt thereof to the subject.

### C. Dosage and Effective Amounts

Dosage and dosing regimens are dependent on the severity of the disorder and/or methods of administration, and can be determined by those skilled in the art. A therapeutically effective amount of GLP-1r agonist, or pharmaceutical formulation thereof used in the treatment of neurological impairment is typically sufficient to reduce or alleviate one or more symptoms of the neurological condition or disorder.

Preferably, the compositions do not target or otherwise modulate the activity or quantity of healthy cells not within or associated with the diseased or target tissues, or do so at a reduced level compared to target cells including activated microglial cells in the CNS. In this way, by-products and other side effects associated with the compositions are reduced.

Administration of the compositions leads to an improvement, or enhancement, of neurological function in an individual with neurological impairment, or neuronal decline or impairment. In some embodiments, the long-acting GLP-1r agonists are administered to a subject in a therapeutically effective amount to stimulate or induce neural mitosis leading to the generation of new neurons, providing a neurogenic effect. Also provided are effective amounts of the compositions to prevent, reduce, or terminate deterioration, impairment, or death of an individual's neurons, neurites and neural networks, providing a neuroprotective effect.

The actual effective amounts of long-acting GLP-1r agonists can vary according to factors including the specific agent administered, the particular composition formulated, the mode of administration, and the age, weight, condition of the subject being treated, as well as the route of administration and the disease or disorder. In some embodiments, the dose of the long-acting GLP-1r agonist, or pharmaceutical formulation thereof can be from about 0.01 to about 100 mg/kg body weight, from about 0.01 mg/kg to about 10 mg/kg, and from about 0.1 mg to about 5 mg/kg body weight. In other embodiments, the dosage is an absolute amount of a GLP-1r agonist, or pharmaceutical formulation thereof, for a single administration to a subject, such as from about 0.1 mg up to about 100 mg. For example, in some embodiments, the dosage of a GLP-1r agonist, or pharmaceutical formulation thereof is about 0.1 mg, 0.5 mg, 1 mg, 2 mg, 3 mg, 4 mg, 5 mg, 6 mg, 7 mg, 8 mg, 9 mg or 10 mg, or more than 10 mg, for example 20 mg, 30 mg, 40 mg or 50 mg. In an exemplary embodiment, the dosage of GLP-1r agonist is about 5 mg, administered once a week. Generally, for intravenous injection or infusion, the dosage may be lower than for oral administration.

In general, the timing and frequency of administration will be adjusted to balance the efficacy of a given treatment schedule with the side-effects of the given delivery system. Exemplary dosing frequencies include continuous infusion, single and multiple administrations such as hourly, daily, weekly, monthly, or yearly dosing.

The long-acting GLP-1r agonist, or pharmaceutical formulation thereof can be administered daily, biweekly, once weekly, once every two weeks, once every two three weeks, once a month, or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

Dosage can vary, and can be administered in one or more doses daily, once daily, twice weekly, once weekly, once every two weeks, once every two three weeks, once a month, or less frequently. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

In some embodiments, the compositions are administered to a subject for prolonged periods, months to years. In one embodiment, the effects of treatment last for at least 1 year.

In preferred embodiments, the GLP-1r agonist is administered orally to an adult (assuming average 70 kg) in an amount between about 1 mg and about 50 mg, inclusive, preferably between about 5 mg and about 20 mg, inclusive. In some embodiments, the GLP-1r agonist is administered orally once a month, once every two weeks, once a week, once every three days, once every two days, once daily, or twice daily.

In other preferred embodiments, the GLP-1r agonist is administered parentally at a concentration between about 0.1 mg/mL and about 10 mg/mL, inclusive, preferably between about 1 mg/mL and about 5 mg/mL, inclusive. In some embodiments, the GLP-1r agonist is administered parentally once a month, once every two weeks, once a week, once every three days, once every two days, or once a day.

In some embodiments, the regimen includes one or more cycles of a round of therapy followed by a drug holiday (e.g., no drug). The drug holiday can be 1, 2, 3, 4, 5, 6, or 7 days, or 1, 2, 3, 4 weeks, or 1, 2, 3, 4, 5, or 6 months.

In some embodiments, the amount of a long-lasting GLP-1r agonist administered to a subject changes over time following an initial dose. Therefore, in some embodiments, the amount of GLP-1r agonist administered to a subject changes over time following an initial dose. For example, in some embodiments, the dose of GLP-1r agonist is titrated with weekly increases from about 1.0 mg up to about 50 mg, inclusive, or about 2.5 mg up to about 20 mg, inclusive.

In some embodiments, the compositions are administered in an amount effective to treat or prevent one or more symptoms of Alzheimer's Disease. In some embodiments, the compositions are administered in an amount effective to reduce or prevent one or more of the clinical markers of Alzheimer's Disease. In some embodiments, the compositions are administered in an amount effective to treat or prevent one or more symptoms of Parkinson's Disease. In some embodiments, the compositions are administered in an amount effective to reduce or prevent one or more of the clinical markers of Parkinson's Disease.

### D. Combination Therapies and Procedures

The compositions can be administered alone or in combination with one or more conventional therapies. Examples of preferred additional therapeutic agents include other conventional therapies known in the art for treating the desired disease, disorder, or condition. The additional therapeutic, prophylactic or diagnostic agent(s) can have the same or different mechanisms of action. In some embodiments, the combination results in an additive effect on the treatment of the disease or condition. In some embodiments, the combinations result in a more than additive effect on the treatment of the disease or disorder.

In the context of Alzheimer's disease, the other therapeutic agents can include one or more of acetylcholinesterase inhibitors (such as tacrine, rivastigmine, galantamine or donepezil), beta-secretase inhibitors such as JNJ-54861911, antibodies such as aducanumab, agonists for the 5-HT2A receptor such as pimavanserin, sargramostim, AADvac1, CAD106, CNP520, gantenerumab, solanezumab, and memantine.

In the context of Parkinson's disease, the conventional treatment can include levodopa (usually combined with a dopa decarboxylase inhibitor or COMT inhibitor), a dopamine agonist, or an MAO-B inhibitor.

Exemplary neuroprotective agents include, for example, glutamate antagonists, antioxidants, and NMDA receptor stimulants. Other neuroprotective agents and treatments include caspase inhibitors, trophic factors, anti-protein aggregation agents, and therapeutic hypothermia.

### E. Controls

The therapeutic result of the long-acting GLP-1r agonist, or pharmaceutical formulation thereof, can be compared to a control or reference. The terms “control” or “reference” refer to a standard of comparison. The term “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result. Suitable controls are known in the art and include, for example, an untreated subject or untreated cells or the same individual prior to treatment.

### V. Kits

The compositions can be packaged in kit. The kit can include a single dose or a plurality of doses of a composition including one or more of the long-acting GLP-1r agonists, or pharmaceutical formulation thereof, and instructions for administering the compositions. In preferred embodiments, the long-acting GLP-1r agonists have the amino acid sequence of any one of SEQ ID NOs: 1-35. Specifically, the instructions direct that an effective amount of the composition be administered to an individual with a particular symptoms, neurological disease, defect, or impairment as indicated. The composition can be formulated as described above with reference to a particular treatment method and can be packaged in any convenient manner.

The present invention will be further understood by reference to the following non-limiting examples.

### EXAMPLES

### Example 1: GLP-1r Agonists with Modifications

GLP-1r agonists were prepared based on the amino acid sequences shown in Table 1. Modifications such as addition of biotin and/or fatty acid chains at various positions of the GLP-1r agonists are listed in Table 5.

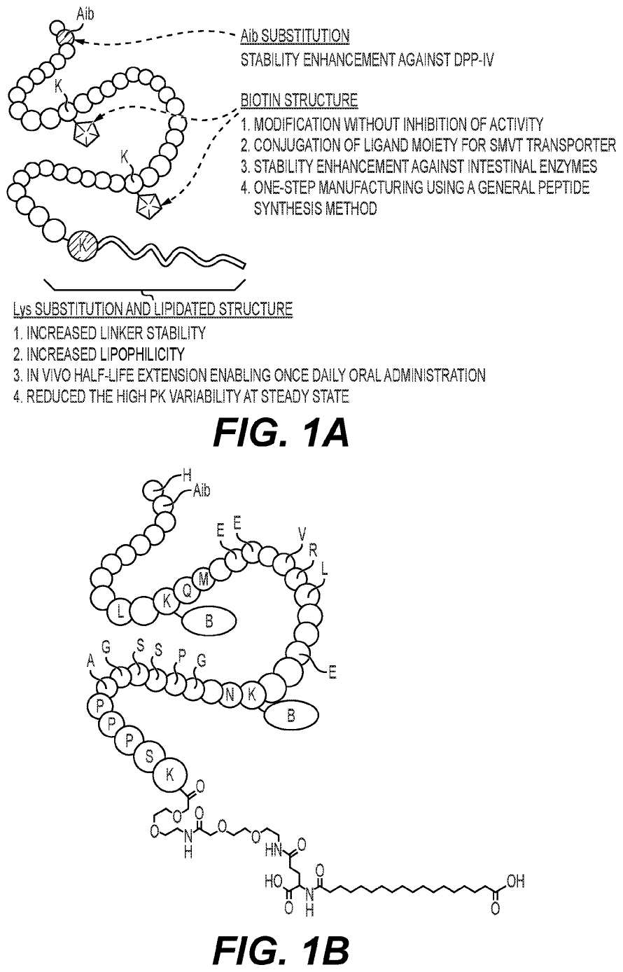

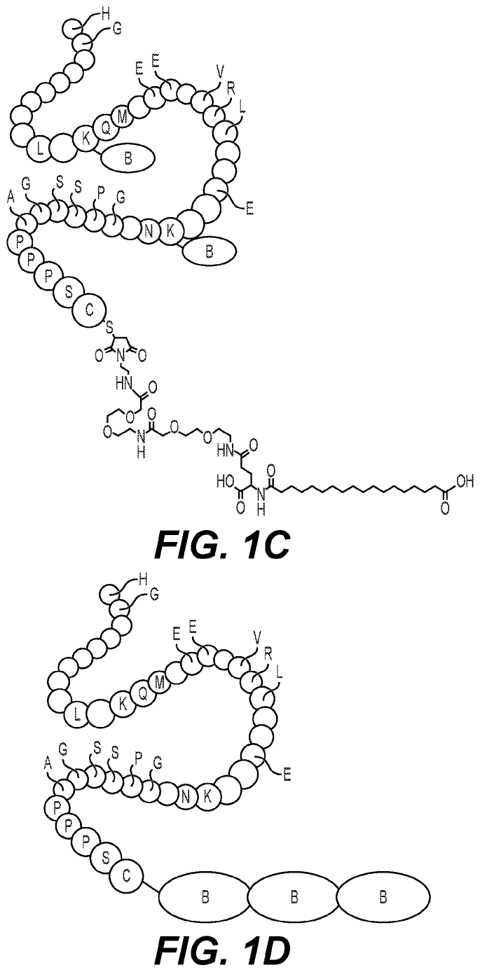

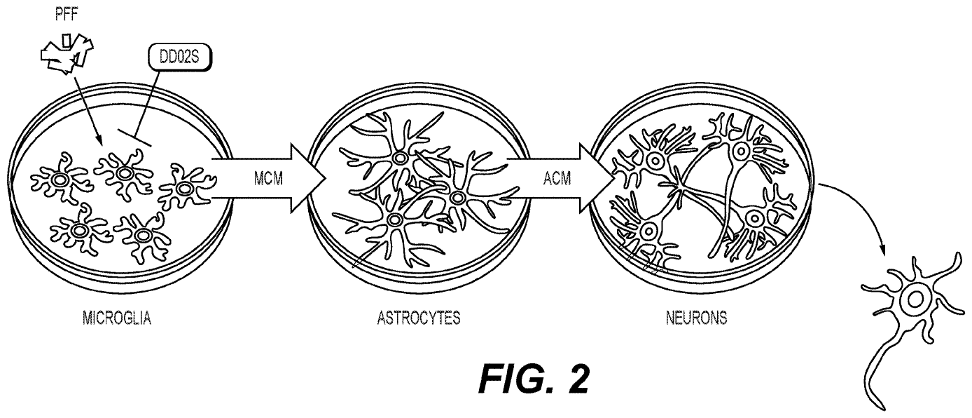

FIG. 1A is a schematic showing exemplary Aib substitution at amino acid position 2, exemplary addition of biotin moieties to Lysine residues at positions 12 and 27, and exemplary addition of a lipid moiety to C-terminal amino acid Lysine at position 40.

SEQ ID NO:29 (also referred to as DD02S) has the same amino acid sequence as SEQ ID NO:5: H-Aib-EGTFTSDLSKQMEEEAVRLFIEWLKNGGPSSGAPPPSK, with a biotin moiety conjugated to each Lysine at positions 12 and 27, and a fatty acid moiety (e.g., 20EG-γGlu-C18) conjugated to C-terminal amino acid Lysine at position 40. A schematic illustration of a GLP-1r agonist having SEQ ID NO:29 is shown in FIG. 1B.

SEQ ID NO:17 (also referred to as DD0207) has the same amino acid sequence as SEQ ID NO:2: HGEGTFTSDLSKQMEEEAVRLFIEWLKNGGPSSGAPPPSC, with a biotin moiety conjugated to each Lysine at positions 12 and 27, and a fatty acid moiety (e.g., 20EG-γGlu-C18) conjugated to C-terminal amino acid Cysteine at position 40. A schematic illustration of a GLP-1r agonist having SEQ ID NO:17 is shown in FIG. 1C.

SEQ ID NO:30 has the same amino acid sequence as SEQ ID NO:7: H-Aib-EGTFTSDLSKQMEEEAVRLFIEWLKNGGPSSGAPPPSC, with a biotin moiety conjugated to each Lysine at positions 12 and 27, and a fatty acid moiety (e.g., 20EG-γGlu-C18) conjugated to C-terminal amino acid Cysteine at position 40.

SEQ ID NO:31 has the same amino acid sequence as SEQ ID NO:8: h-Aib-EGTFTSDLSKQMEEEAVRLFIEWLKNGGPSSGAPPPSC, with a biotin moiety conjugated to each Lysine at positions 12 and 27, and a fatty acid moiety (e.g., 20EG-γGlu-C18) conjugated to C-terminal amino acid Cysteine at position 40.

SEQ ID NO:11 has the same amino acid sequence as SEQ ID NO:2: HGEGTFTSDLSKQMEEEAVRLFIEWLKNGGPSSGAPPPSC, with three biotin moieties conjugated to C-terminal amino acid Cysteine at position 40. A schematic illustration of a GLP-1r agonist having SEQ ID NO:11 is shown in FIG. 1D.

SEQ ID NO:20 has the same amino acid sequence as SEQ ID NO:3: HGEGTFTSDLSKQMEEEAVRLFIEWLKNGGPSSGAPPPSK, with three biotin moieties conjugated to C-terminal amino acid Lysine at position 40 (e.g., K40-Ac-B3-PEG2).

SEQ ID NO:24 has the same amino acid sequence as SEQ ID NO:5: H-Aib-EGTFTSDLSKQMEEEAVRLFIEWLKNGGPSSGAPPPSK, with three biotin moieties conjugated to C-terminal amino acid Lysine at position 40.

### Example 2: DD02S Inhibits Microglial Activation Induced by α-Syn PFF and Inhibits Activation of Reactive Astrocytes by MCM Treated with α-Syn PFF

### Materials and Methods

### Primary Microglia and Astrocyte Cell Cultures

Mouse microglia isolated from postnatal day 2 CD1 mouse brain were obtained from ScienCell Research Laboratories (M1900, Carlsbad, CA). For qPCR analysis, 3×10⁵ of microglia were seeded onto 12 well cell culture plates coated with Poly-L-Lysine (1 mg/ml) (0403 ScienCell Research Laboratories) and were maintained in microglia medium (1901, ScienCell Research Laboratories), until confluent and ready to use. Pre-condition: 24 hrs non-serum condition. Experimental culture condition: NLY01, Semaglutide, DD02S: 2 μM, 150 nM PFF for 4 h.

Mouse astrocytes were obtained from ScienCell Research Laboratories (M1800) and maintained in growth medium (ScienCell Catalog #1831). Pre-condition: 24 hrs non-serum condition. Experimental culture condition: MCM treated with NLY01, Semaglutide, DD02S: 1 μM, followed by PFF 150 nM for 24 h. For MCM collection, 106 of microglia were seeded onto 6 well cell culture plates coated with PLL and were maintained in microglia medium until confluent and ready to use.

The conditioned medium from the primary microglia treated with α-syn PFF (α-syn PFF-MCM) with either PBS or indicated treatment were collected and concentrated with Amicon Ultra-15 centrifugal filter unit (30 kDa cutoff) (Millipore) until approximately 10× concentrated, and then applied to primary astrocytes for 24 h.

Comparative qPCR

Total RNA from cultured cells was extracted with a RNA isolation kit (Qiagen, Valencia, CA, USA) following the instructions provided by the company. RNA concentration was measured spectrophotometrically using a NanoDrop 2000 (Biotek, Winooski, VT, USA). Subsequently, 1-2 μg of total RNA was reverse transcribed to cDNA using the High-Capacity cDNA Reverse Transcription System (Life Technologies, Grand Island, NY, USA). Comparative qPCR was performed in duplicate or triplicate for each sample using fast SYBR Green Master Mix (Life Technologies) and ViiA 7 Real-Time PCR System (Applied Biosystems, Foster City, CA, USA). The expression levels of target genes were normalized to the expression of Actb and calculated based on the comparative cycle threshold Ct method (2^{-ΔΔCt}). Sequences of primers are as described by Yun, S. P. et al., Nat Med 24, 931-938 (2018).

### Results

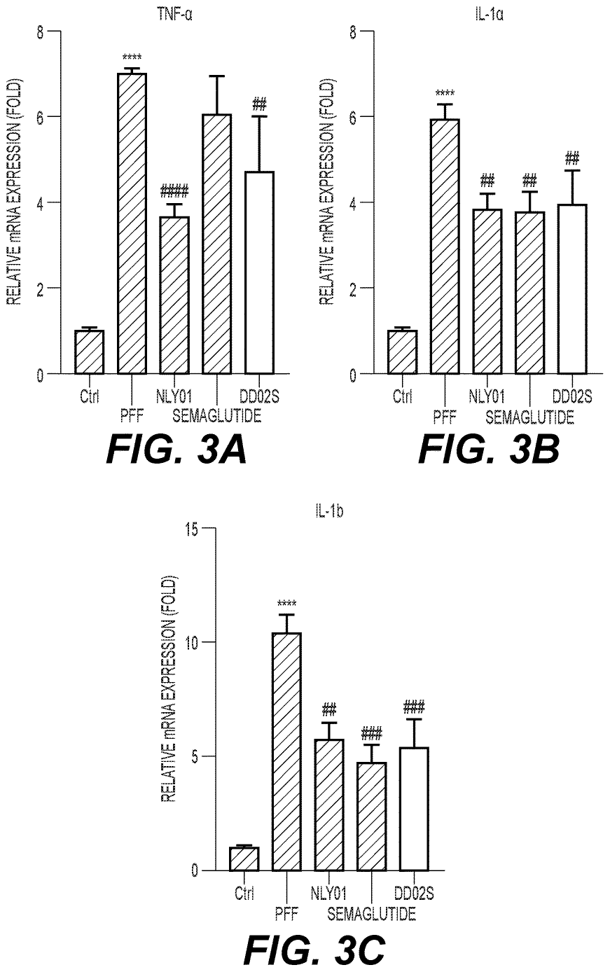

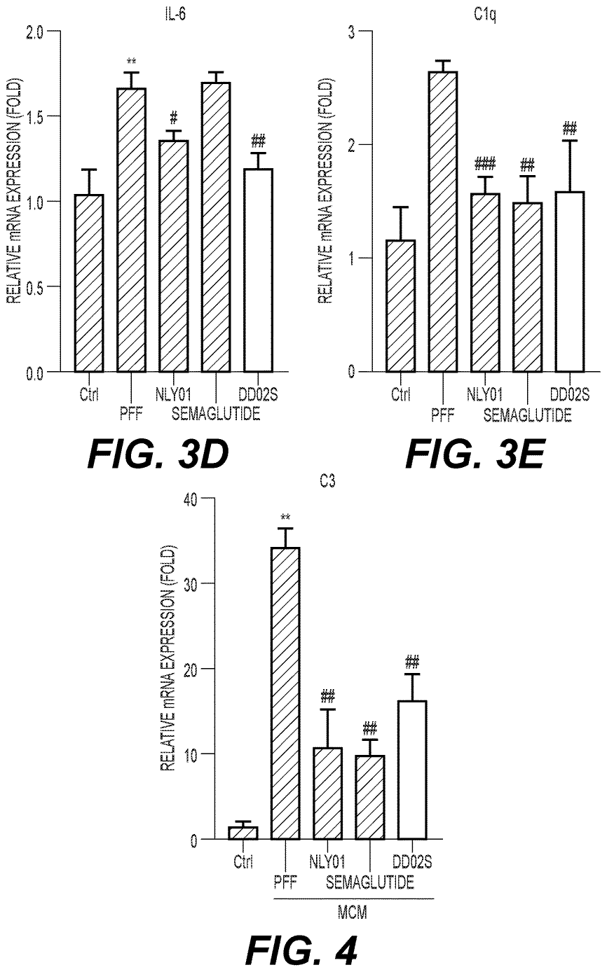

To further investigate the ability of α-synuclein preformed fibril (α-syn PFF) to activate microglia and the inhibitory effect of the GLP-1r agonists, the levels of TNF-α, IL-1α, IL-1β, C1q and IL-6 were monitored in response to α-syn PFF (FIGS. 3A-3E). α-syn PFF significantly induces mRNA expression of TNF-α, IL-1α, IL-1β, C1q and IL-6 as determined by quantitative PCR (qPCR) in primary microglia. The GLP-1r agonists DD02S significantly reduces this mRNA induction, like the control GLP-1r agonists NLY01 and Semaglutide.

Since NLY01 preferentially blocks microglia-derived factors that induce reactive astrocytes, α-syn PFF microglial-conditioned medium (MCM) was applied to astrocytes for 24 h. The GLP-1r agonist, DD02S, also inhibits the reactive astrocytes activated by MCM treated with PFF as determined by qPCR (FIG. 4).

### Example 3: DD02S Inhibits Microglial Activation Induced by β-Amyloid Oligomer (ABO) and Inhibits Activation of Reactive Astrocytes by MCM Treated with ABO

### Materials and Methods

### Primary Microglia and Astrocyte Cell Cultures

Mouse microglia isolated from postnatal day 2 CD1 mouse brain were obtained from ScienCell Research Laboratories (M1900, Carlsbad, CA). For qPCR analysis, 3×10⁵ of microglia were seeded onto 12 well cell culture plates coated with Poly-L-Lysine (1 mg/ml) (0403 ScienCell Research Laboratories) and were maintained in microglia medium (1901, ScienCell Research Laboratories), until confluent and ready to use. Pre-condition: 24 hrs non-serum condition. Experimental culture condition: NLY01, Semaglutide, DD02S: 1μ, 1μ M AβO for 4 h.

Mouse astrocytes were obtained from ScienCell Research Laboratories (M1800) and maintained in growth medium (ScienCell Catalog #1831). Pre-condition: 24 hrs non-serum condition. Experimental culture condition: MCM treated with NLY01, Semaglutide, DD02S: 1 μM, followed by 1 μM ABO for 24 h. For MCM collection, 106 of microglia were seeded onto 6 well cell culture plates coated with PLL and were maintained in microglia medium until confluent and ready to use.

The conditioned medium from the primary microglia treated with ABO (ABO-MCM) with either PBS or indicated treatment was collected and concentrated with Amicon Ultra-15 centrifugal filter unit (30 kDa cutoff) (Millipore) until approximately 10× concentrated, and then applied to primary astrocytes for 24 h.

### Results

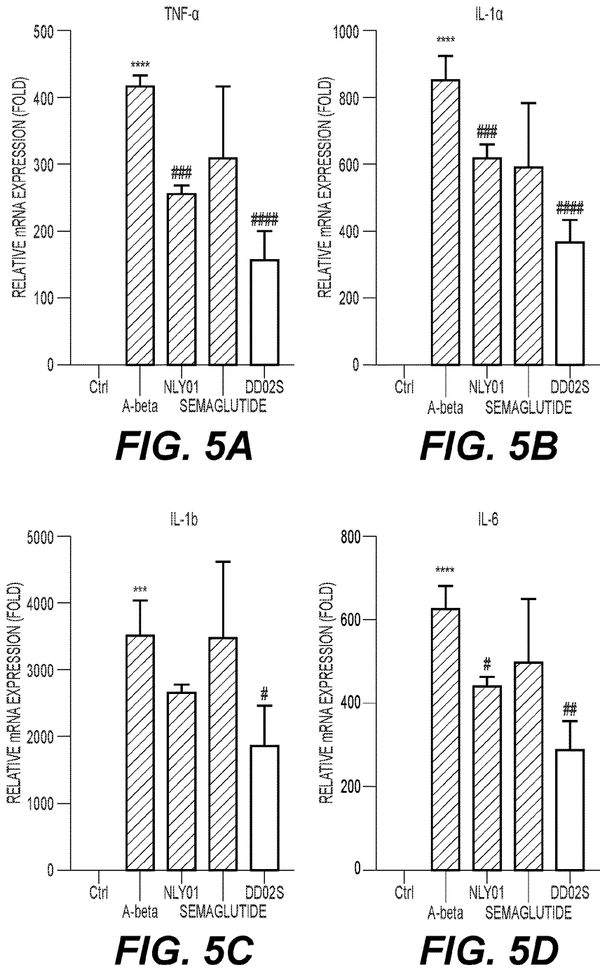

To investigate the inhibitory effect on activation of microglia by the GLP-1r agonists, the levels of TNF-α, IL-1α, IL-1β, and IL-6 were monitored in response to AβO. AβO significantly induces mRNA expression of TNF-α, IL-1α, IL-1β, and IL-6 as determined by quantitative PCR (qPCR) in primary microglia (FIGS. 5A-5D). The GLP-1r agonist DD02S significantly reduces the induced mRNA expression of TNF-α, IL-1α, IL-1β, and IL-6. Thus, DD02S inhibits microglial activation induced by AβO.

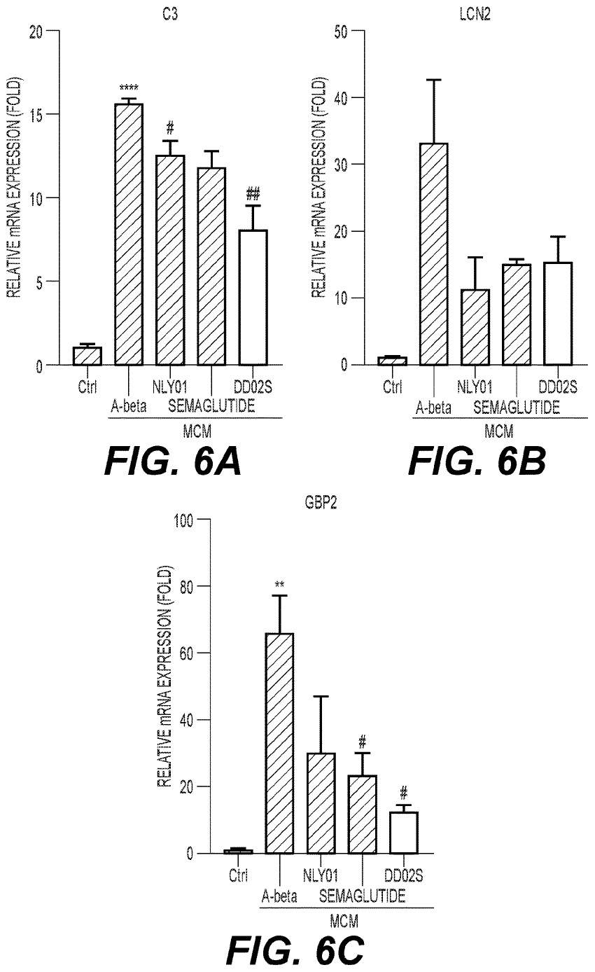

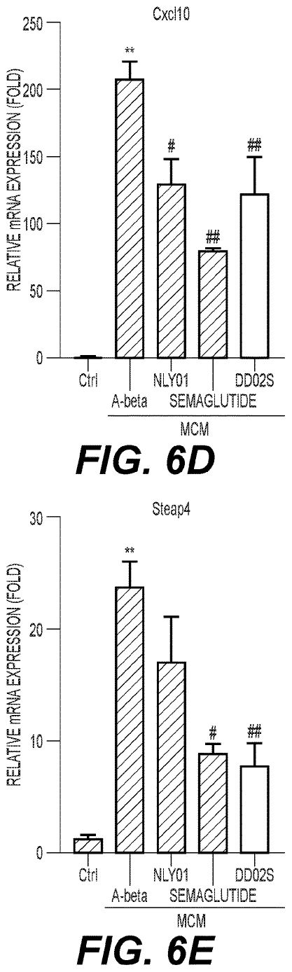

To examine the mechanism of action by the GLP-1r agonists, AβO microglial-conditioned medium (MCM) was applied to astrocytes for 24 h. ABO significantly induces mRNA expression of C3, LCN2, GBP2, Cxc110, and Steap4 (FIGS. 6A-6E). However, this induction was significantly inhibited by the GLP-1r agonist, DD02S. Thus, DD02S also inhibits the reactive astrocytes activated by MCM treated with ABO.

### Example 4: Additional GLP-1r Agonists Inhibit Microglial Activation Induced by β-Amyloid Oligomer (AβO)

### Materials and Methods

To investigate the inhibitory effect on activation of microglia by additional GLP-1r agonists, the levels of TNF-α, IL-1α, IL-1β, C1q, and IL-6 were monitored in response to ABO following a 30-min pretreatment with the GLP-1r agonists.

### Results

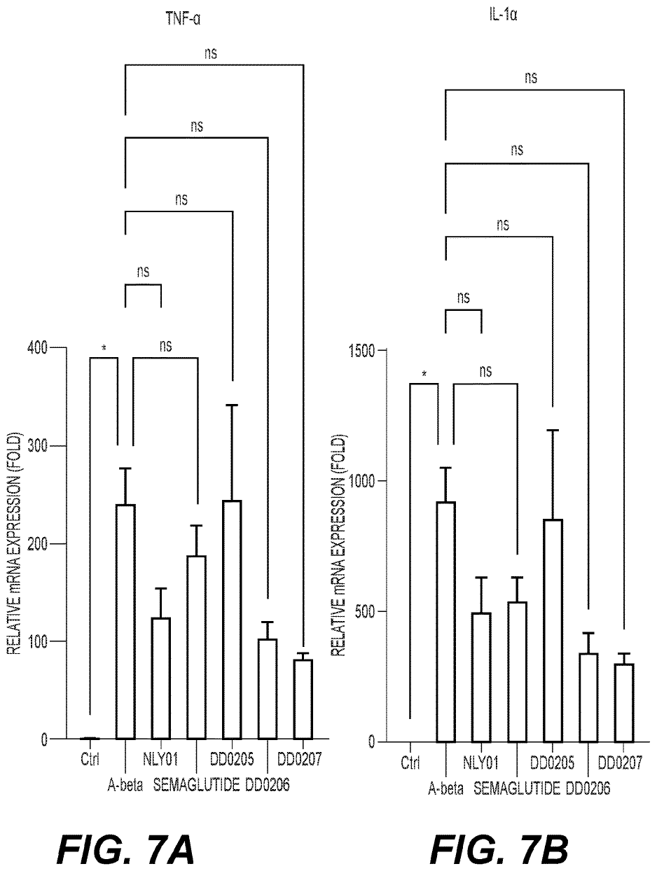

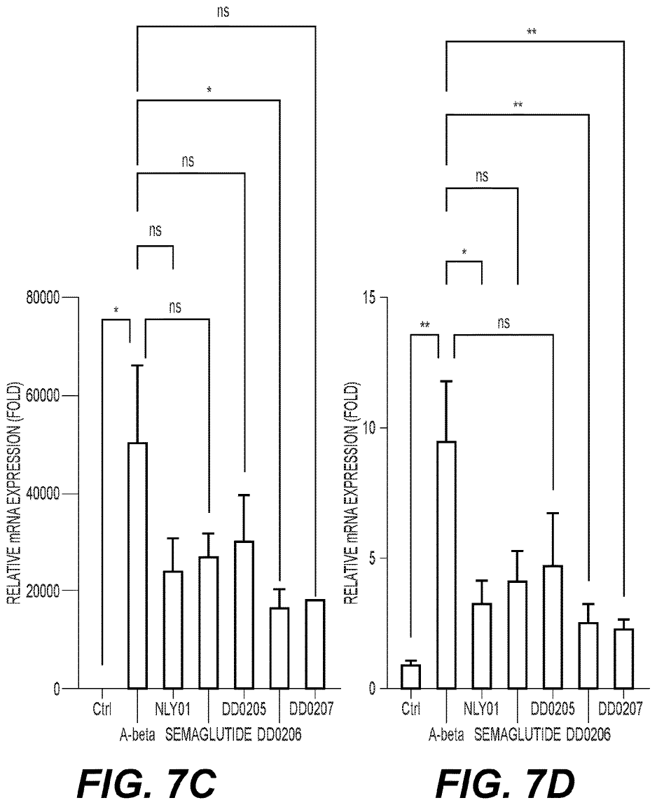

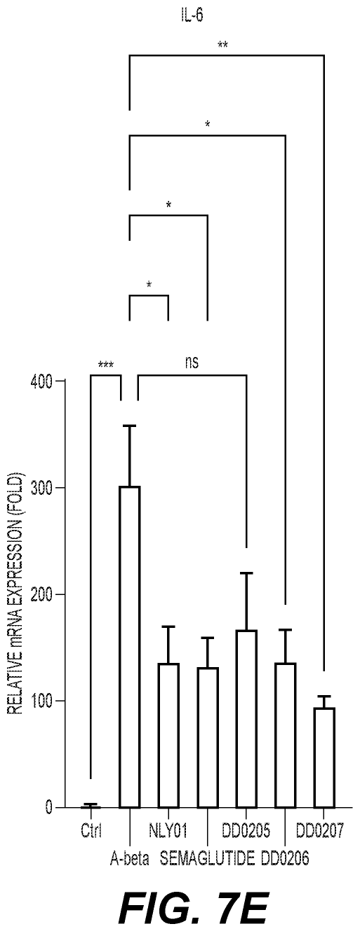

AβO significantly induces mRNA expression of TNF-α, IL-1α, IL-1β, C1q, and IL-6 as determined by quantitative PCR (qPCR) in primary microglia (FIGS. 7A-7E).

GLP-1r agonists DD0205 (SEQ ID NO:15), DD0206 (SEQ ID NO: 16), an DD0207 significantly reduces the induced mRNA expression of C1q and IL-6. Thus, DD0205, DD0206, and DD0207 can inhibit microglial activation induced by ABO.

### Example 5: SMVT Mediated Dose Dependent Intracellular Uptake of Biotinylated GLP-1r Agonists is Confirmed in Caco-2 Cells

### Materials and Methods

SMVT is an important transmembrane protein responsible for translocation of vitamins and other essential cofactors and highly expressed in human tissues such as intestine, brain, liver, lung, retina and heart. Intracellular uptake of biotinylated peptides was evaluated in human intestinal cells, Caco-2 cells.

### Results

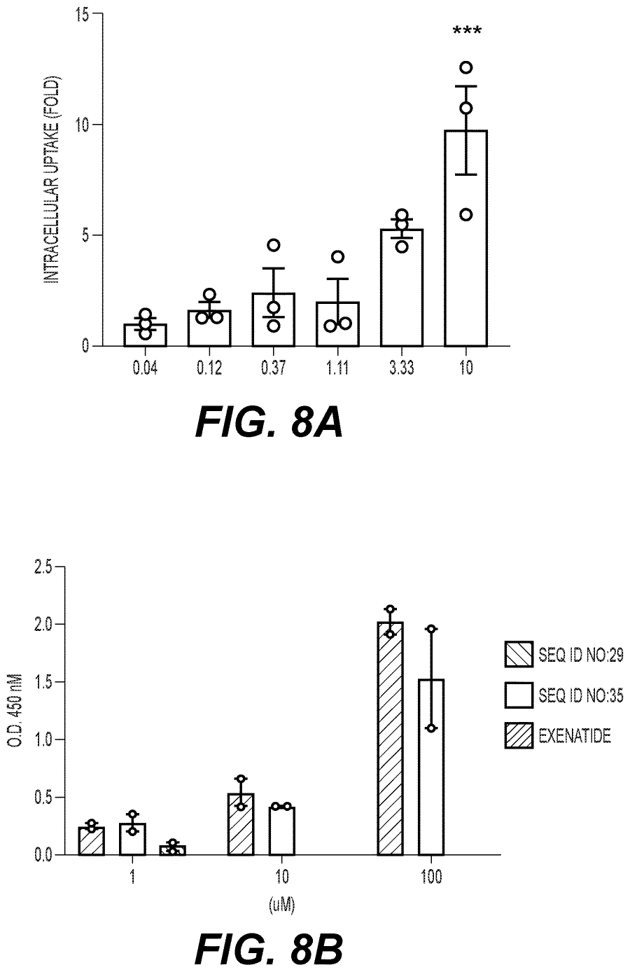

Intracellular uptake of Exenatide with very low uptake degree is enhanced by biotin modification (FIGS. 8A and 8B).

### Example 6: DD02S Shows the Superior Stability Against Gastrointestinal Enzymes Such as Trypsin and FaSSIF/Pancreatin

### Materials and Methods

To determine the stability of DD02S in the gastrointestinal environment, the in vitro stability against trypsin and fasted state simulated intestinal fluid/pancreatin (FaSSIF/pancreatin) of DD02S was checked and the enzymatic stability of DD02S, exenatide and semaglutide was compared.

### Results

DD02S was more stable than Exenatide and Seamglutide against trypsin and FaSSIF/pancreatin (Table 6 and Table 7).

TABLE 6
Remaining amount (%) of GLP-1r agonists after trypsin treatment

| Time (min) | DD02S | Exenatide | Semaglutide |
|---|---|---|---|
| 0 | 100.0 | 100.0 | 100.0 |
| 10 | 80.5 | N.D. | N.D. |
| 30 | 60.4 | — | — |
| 60 | 41.4 | N.D. | N.D. |
| 90 | 29.0 | — | — |
| 120 | 20.5 | — | — |
| 180 | 11.4 | N.D. | N.D. |
| 360 | 2.9 | — | — |

*N.D.: not detected

TABLE 7
Remaining amount (%) of GLP-1r agonists
after FaSSIF/Pancreatin treatment

| Time (min) | DD02S | Exenatide | Semaglutide |
|---|---|---|---|
| 0 | 100.0 | 100.0 | 100.0 |
| 10 | 69.8 | 44.3 | 49.1 |
| 30 | 48.2 | 15.3 | 4.6 |
| 60 | 25.2 | 0.8 | N.D. |
| 120 | 6.1 | N.D. | N.D. |
| 180 | 1.9 | N.D. | N.D. |

*N.D.: not detected

### Example 7: DD02S Shows the Higher Oral Bioavailability after Administration of Enteric Tablets in Beagle Dogs

### Materials and Methods

To evaluate and compare the oral absorption of DD02S, the male beagle dogs were dosed orally with a DD02S tablet (10 mg) and a RYBELSUS® (14 mg), which is an approved oral GLP-1r agonist.

### Results

DD02S tablet showed the higher Cmax, AUC and bioavailability than RYBELSUS® (Table 8).

TABLE 8
Pharmacokinetic profiles of DD02S tablet and RYBELSUS ® in beagle dogs

| | | Half- | | | | |
|---|---|---|---|---|---|---|
| | API dose | life | Cmax | AUC_{120 h} | AUC_{120 h}/dose | Bioavailability |
| | (mg/tablet) | (hr) | (nM) | (h · nmol/L) | (h · kg/L) | (%) |
| DD02S | 10 mg | 57 ± 10 | 100 ± 77 | 4,563 ± 3,564 | 21.5 ± 16.7 | 5.0 ± 3.9 |
| RYBELSUS ® | 14 mg | 41 ± 19 | 65 ± 82 | 885 ± 1,045 | 2.5 ± 3.0 | 0.4 ± 0.5 |

### Example 8: DD02S Reduces the Level of α-Syn in the Brain of α-Syn PFF Induced PD Animal Models

### Materials and Methods

### Animal Models

C57BL6 mice (male, 7-week-old) were obtained from the Orient Bio Co. Ltd. (Seongnam, Korea). Alpha-synuclein Preformed Fibril (α-syn PFF)-induced PD mice were prepared. α-syn is a key protein involved in PD pathology. For stereotaxic injection of α-syn PFF, 12 week-old male mice were anesthetized with Avertin. An injection cannula (26.5 gauge) was applied stereotaxically into the striatum (anteroposterior, 0.2 mm from bregma; mediolateral, 1.2 mm; dorsoventral, 2.6 mm) or unilaterally (applied into the right hemisphere). The infusion was performed at a rate of 0.2 μL per min, and 2 μL of α-syn PFF (5 μg/mL in PBS) or same volume of PBS were injected into mouse. The head skin was closed by suturing and wound healing and recovery were monitored following surgery.

Four weeks after α-syn PFF stereotaxic injection, DD02S was subcutaneously (0.61 mg/kg) or orally (11 mg/kg) administered once daily for 10 weeks (n=10). Subcutaneous doses were administered via bolus injection between the skin and underlying layers of tissue in the scapular region on the back of each animal. For oral dosing, the dose formulation was administered by oral gavage. The dose formulation for subcutaneous injection was the appropriate amount of DD02S in 10 mM PBS containing polysorbate 80. The dose formulation for oral gavage was 1.1 mg/mL of DD02S, sodium chenodeoxycholate, sodium ursodeoxycholate, and 6 propyl gallate in 10 mM PBS containing polysorbate 80. Animals were weighed prior to dose administration and dose volume were 10 ml/kg.

### Immunohistochemistry and Quantitative Analysis

For stereological analysis, animals were perfused and fixed intracardially with ice-cold PBS followed by 4% paraformaldehyde 14 weeks after striatal α-syn PFF injection. The brain was removed and processed for immunohistochemistry. The immunohistochemistry tests were conducted in vehicle (PBS) or DD02S treated mice at 14 weeks after post α-synuclein PFFs injection. Immunohistochemistry (IHC) was performed on 30 μm thick serial brain sections. Free-floating 30 μm sections were blocked with 30% goat serum/PBS plus 0.1% Triton X-100. Sections stained with phospho-serine 129 α-synuclein (pS129-α-syn, Abcam, UK) using Mouse and Rabbit Specific HRP/DAB IHC Detection Kit-Micro-polymer kit (Abcam. ab236466) following user manual and counterstained with Nissl (0.09% thionin). The number of immune-reactive positive cells in the Striatum (STR) and Substantia Nigra Pars Compacta (SNpc) region were measured with ImageJ software.

### Results

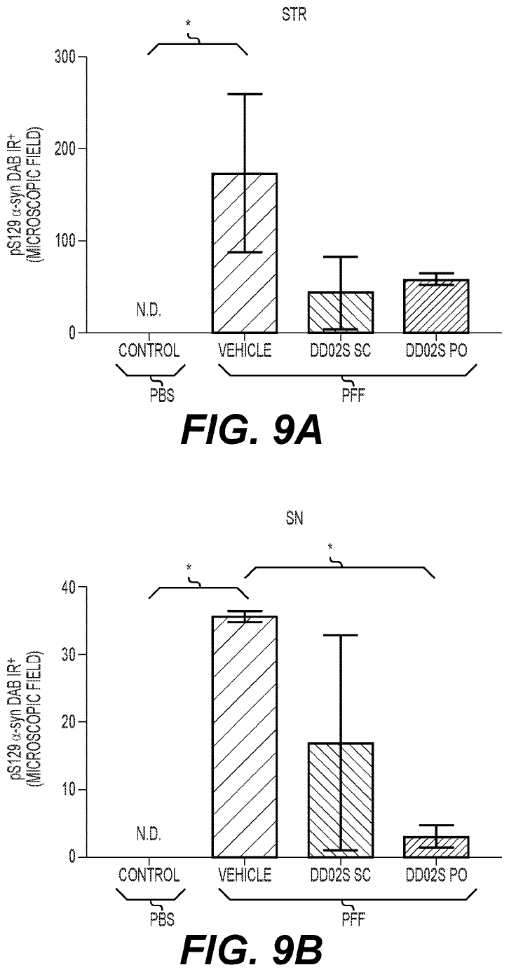

To validate the efficacy of DD02S in α-syn PFF-induced PD pathology, pS129-α-syn immunoreactivity was monitored in the STR and SNpc of mice at three months after α-syn PFF intrastriatal injection. There was substantial pS129-α-syn immunoreactivity in the STR and SNpc of α-syn PFF-injected mice. Treatment of DD02S via subcutaneous injection (0.61 mg/kg) and oral gavage (11 mg/kg) reduced pS129-α-syn immunoreactivity in the STR (Table 9 and FIG. 9A) and SNpc (Table 10 and FIG. 9B). DD02S reduced the Lewy body-like pathology in brain of α-syn PFF-induced PD animal models.

TABLE 9
Quantification of immunoreactivity for pS129-α-syn in the STR

| | | PFF | PFF |
|---|---|---|---|
| SHAM | PFF | DD02S | DD02S |
| Vehicle | Vehicle | (Subcutaneous) | (Oral) |
| N.D. | 173.75 ± 85.75 | 43.87 ± 39.87 | 58.75 ± 6.25 |

*N.D.: not detected

TABLE 10
Quantification of immunoreactivity for pS129-α-syn in the SNpc

| | | PFF | PFF |
|---|---|---|---|
| SHAM | PFF | DD02S | DD02S |
| Vehicle | Vehicle | (Subcutaneous) | (Oral) |
| N.D. | 35.75 ± 0.75 | 17 ± 16 | 3.167 ± 1.691 |

*N.D.: not detected

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A method of treating or preventing a neurodegenerative disease or disorder in a subject suffering from or at risk of developing a neurodegenerative disease or disorder, comprising
administering a pharmaceutically effective amount of a composition comprising a long-acting GLP-1r agonist to treat or alleviate one or more symptom of the neurodegenerative disease or disorder.

2. The method of claim 1, wherein the long-acting GLP-1r agonist comprises
(i) a polypeptide comprising the amino acid sequence of any one of SEQ ID NOs: 1-8; and
(ii) one or more biotin moieties and/or one or more fatty acids, or derivatives thereof,
wherein the one or more biotin moieties and/or one or more fatty acids, or derivatives thereof are conjugated to the polypeptide.

3. The method of claim 1, wherein the one or more biotin moieties and/or one or more fatty acids, or derivative thereof, are conjugated to the polypeptide via one or more amino acid residues selected from the group consisting of cysteine and lysine.

4. The method of claim 1, wherein the one or more biotin moieties and/or one or more fatty acids, or derivative thereof, are conjugated to the polypeptide via one or more amino acid residues selected from the group consisting of lysine at position 12, lysine at position 27, and a C-terminal residue.

5. The method of claim 1, wherein the long-acting GLP-1r agonist comprises the amino acid sequence of any one of SEQ ID NOs: 9-35.

6. The method of claim 1, wherein the composition is administered in an amount effective to inhibit the secretion of inflammatory and/or neurotoxic mediators secreted from activated microglial cells, as compared to an appropriate control.

7. The method of claim 1, wherein the composition is administered in amount effective to inhibit or reduce the secretion of inflammatory and/or neurotoxic mediators secreted from astrocytes, as compared to an appropriate control.

8. The method of claim 1, wherein the composition is administered in an effective amount to reduce one or more inflammatory or neurotoxic mediators in the subject as compared to an appropriate control, wherein the inflammatory or neurotoxic mediators are selected from the group consisting of TNF-α, IL-1α, IL-1β, IFN-γ, IL-6, and C1q.

9. The method of claim 1, wherein the composition is administered in an effective amount to reduce the number of activated microglial cells in the brain of the subject.

10. The method of claim 1, wherein the composition is administered in an effective amount to reduce abnormally aggregated proteins through upregulation of GLP-1r.

11. The method of claim 10, wherein the abnormally aggregated proteins are α-synuclein, β-amyloid or tau.

12. The method of claim 1, wherein the neurodegenerative disease or disorder is Parkinson's disease.

13. The method of claim 1, wherein the neurodegenerative disease or disorder is Alzheimer's disease.

14. The method of claim 1, wherein the composition is administered via a route selected from the group consisting of enteral administration and parenteral administration.

15. The method of claim 1, wherein the composition is administered via oral administration or subcutaneous administration.

16. The method of claim 1, wherein the composition is administered in a form selected from the group consisting of pills, capsules, tablets, liquids, and suspensions.

17. The method of claim 1, wherein the composition is administered at an interval selected from the group consisting of once a month, once every two weeks, once a week, once every three days, once every two days, once daily, and twice daily.

18. The method of claim 1, wherein the composition is administered to the subject once a week for up to 6 months.

19. The method of claim 1, wherein the composition is administered to the subject for a duration of between one and ten days, weeks, months, or years, inclusive.

20. The method of claim 1, wherein the composition is administered to a human subject at a dose of between about 0.001 mg/kg body weight of the subject and about 100 mg/kg body weight of the subject, inclusive.

21. The method of claim 1, wherein the composition is administered to a human subject at a dose of between 1.0 mg and 100 mg, inclusive.
